(19) United States
(12) Patent Application Publication
(10) Pub. No.: US 2026/0338772 A1
(43) Pub. Date: Oct. 29, 2026

(54) DEVICES FOR DISPENSING AN IMMUNOREACTIVE MAGNETIC PARTICLE AND IMMUNOASSAY REPORTER IN A FLUID DROPLET

(71) Applicant: Hewlett-Packard Development Company, L.P., Spring, TX (US)

(72) Inventors: Viktor Shkolnikov, Palo Alto, CA (US); Michael Cumbie, Corvallis, OR (US)

(21) Appl. No.: 19/106,363

(22) PCT Filed: Sep. 15, 2022
(86) PCT No.: PCT/US2022/043621

§ 371 Date: Feb. 25, 2025

(51) Int. Cl. G01N 33/543

(52) U.S. Cl. CPC G01N 33/54386; G01N 33/54326

(57) ABSTRACT

An example device includes a substrate, a microfluidic network supported by the substrate, a magnet integrated with the substrate within a region of the microfluidic channel downstream from the sample fluid channel and the reporter fluid channel, and a droplet ejector coupled to a distal end of the microfluidic channel. The microfluidic network includes a microfluidic channel, a sample fluid channel for delivering a sample fluid to the microfluidic channel, and a reporter fluid channel for delivering a fluid comprising an immunoassay reporter to the microfluidic channel. The sample fluid includes an immunoreactive magnetic particle. The magnet is positioned adjacent to a wall of the microfluidic channel. The droplet ejector is for dispensing a fluid droplet comprising the immunoreactive magnetic particle and the immunoassay reporter.

### BACKGROUND

Immunoassays are biochemical tests that detect the presence or concentration of a target molecule in a sample using an antibody. One immunoassay format is the enzyme-linked immunoassay (ELISA). The sandwich ELISA has the highest sensitivity among all the ELISA types. ELISAs may be used in many settings, including rapid antibody screening tests, detection of viruses, bacteria, fungi, autoimmune diseases, food allergens, laboratory and clinical research, and forensic toxicology, for example.

### BRIEF DESCRIPTION OF THE DRAWINGS

FIG. 1A illustrates an example device for dispensing an immunoreactive magnetic particle and immunoassay reporter in a fluid droplet, in accordance with examples of the present disclosure.

FIG. 1B is a flow diagram of an example method for dispensing an immunoreactive magnetic particle and immunoassay reporter in a fluid droplet, in accordance with examples of the present disclosure.

FIG. 2 illustrates another example device for dispensing an immunoreactive magnetic particle and immunoassay reporter in a fluid droplet, in accordance with examples of the present disclosure.

FIG. 3 illustrates another example device for dispensing an immunoreactive magnetic particle and immunoassay reporter in a fluid droplet, in accordance with examples of the present disclosure.

FIG. 4 illustrates another example device for dispensing an immunoreactive magnetic particle and immunoassay reporter in a fluid droplet, in accordance with examples of the present disclosure.

FIG. 5A illustrates an example implementation of a device for dispensing an immunoreactive magnetic particle and immunoassay reporter in a fluid droplet, in accordance with examples of the present disclosure.

FIGS. 5B-5D show images of fluid droplets dispensed as a planar array, in accordance with examples of the present disclosure;

FIG. 6 illustrates another example implementation of a device for dispensing an immunoreactive magnetic particle and immunoassay reporter in a fluid droplet, in accordance with examples of the present disclosure.

FIG. 7 illustrates an example optical system for analysis of dispensed immunoassay droplets, in accordance with examples of the present disclosure.

FIG. 8 is a flow chart of an example method for dispensing an immunoreactive magnetic particle and immunoassay reporter in a fluid droplet, in accordance with examples of the present disclosure.

FIG. 9 is a flow chart of another example method for dispensing a plurality of fluid droplets of an enzyme-linked immunoassay, and monitoring the dispensed droplets, in accordance with examples of the present disclosure.

FIG. 10 is a block diagram of an example controller for dispensing an immunoreactive magnetic particle and immunoassay reporter in a fluid droplet including a processor and associated non-transient computer-readable medium containing instructions for the processor, in accordance with examples of the present disclosure.

FIG. 11 illustrates an example implementation of the example controller for dispensing an immunoreactive magnetic particle and immunoassay reporter in a fluid droplet.

FIG. 12 illustrates another example implementation of the example controller for dispensing an immunoreactive magnetic particle and immunoassay reporter, in accordance with examples of the present disclosure.

FIG. 13 is a block diagram illustrating an example system for dispensing an immunoreactive magnetic particle and immunoassay reporter in a fluid droplet, in accordance with examples of the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Research and clinical laboratories are under pressure to improve immunoassay performance while reducing costs and increasing throughput. The instruments and tools used for immunoassays are transitioning to simpler microfluidic-based devices and systems, including devices and systems with multiplexing capacity. In many instances, to obtain accurate ELISA results, it may be necessary to reduce background signal related to unbound or non-specifically bound conjugated antibody. For example, insufficient washing may result in variation and high background, and thus poor results. The number of incubation and washing steps contributes to the labor-intensiveness of plate-based sandwich ELISA. For maximum sensitivity, specificity, and precision, virtually all unbound reporter enzyme-conjugates may be removed from the solid-phase. The removal process may be affected by various parameters, such as the composition of a wash solution, the mechanism used to introduce the wash solution, the volume of wash solution used, the number of wash cycles, and presence of residual fluid at the end of a wash cycle. Residual fluid refers to the fluid containing unbound antigen or antibody-conjugate that may remain on the surface of the plate. Total aspiration of fluid at any step is undesirable as this may denature the protein and inactivate the enzyme. In general, the fluid force of the wash fluid should be controlled to preserve enzyme activity of specifically bound antibody conjugates. Minimizing the amount of excess antibody or antigen to lower the background noise may consume high volumes of wash fluid and/or add to the number of wash cycles. However, excessive or robust wash cycles may reduce signal strength, and reduce measurement precision. While bead-based immunoassay platforms may provide increased sensitivity of detection due to the increased surface area for antigen binding, beads may be lost during washing steps, thereby lowering the accuracy of the immunoassay.

Devices for dispensing an immunoreactive magnetic particle and immunoassay reporter in a fluid droplet, in accordance with the present disclosure, may reduce the time and labor spent preparing plate-based immunoassays by automating the removal of unbound or non-specifically bound components. Devices in accordance with the present examples may have particular utility for personal and point-of-care clinical diagnostics by reducing the volume of sample and reagents compared to plate-based or prior bead-based ELISA, and/or by improving the speed, accuracy, and sensitivity of the assay, which is advantageous for detection of analytes that are present at low and extremely low concentrations. For example, a device as described below may dispense a fluid droplet containing a single immunoreactive magnetic particle which has captured a single antigen molecule in a volume of fluid that facilitates economical generation of a detectable signal from the immunoassay reporter, and such devices may efficiently dispense millions of fluid droplets organized for multiplexed analysis. Single particle immunoassays may provide may facilitate earlier diagnosis of a medical condition than plate-based immunoassays, for example.

An example device for dispensing an immunoreactive magnetic particle and immunoassay reporter in a fluid droplet, which allows for efficient removal of unbound or non-specifically bound components is described. The term “example” as used throughout this application is by way of illustration, and not limitation. The term “dispense” refers to or includes the release of a specific amount of the fluid droplet. The term “immunoassay reporter” refers to or includes a molecule that generates a detectable signal in the presence of an enzyme-linked immunocomplex. An “enzyme-linked immunocomplex” refers to or includes a complex formed between a target antigen and an enzyme-conjugated antibody, or antibody fragment, having an affinity to bind the target antigen. As used herein, “antigen” refers to and includes a molecule or structure thereof, or particulate matter, capable of specifically binding to the conjugated antibody or fragment thereof. Detection of the generated signal indicates the fluid specimen contains the antigen.

The example device comprises a substrate; a microfluidic network supported by the substrate, the microfluidic network comprising: a microfluidic channel, a sample fluid channel to deliver a sample fluid comprising an immunoreactive magnetic particle to the microfluidic channel, the immunoreactive magnetic particle including a capture antibody to bind to a target antigen present in the sample fluid, and a reporter fluid channel to deliver a fluid comprising an immunoassay reporter to the microfluidic channel; a magnet integrated with the substrate within a region of the microfluidic channel downstream from the sample fluid channel and the reporter fluid channel, wherein the magnet is positioned adjacent to a wall of the microfluidic channel; and, a droplet ejector coupled to a distal end of the microfluidic channel to dispense a fluid droplet comprising the immunoreactive magnetic particle and the immunoassay reporter.

As used herein, the “microfluidic network” or channel thereof includes and/or refers to a path through which a fluid or semi-fluid may pass, which may allow volumes of fluid, such as microliter (μL), nanoliter, picoliter, or femtoliter volumes, to be transported through the microfluidic device. A microfluidic network may be formed of an etched, microfabricated, and/or micromachined portion of the substrate. As used herein, “substrate” refers to or includes the underlying substance of the device. The substrate may be formed of any suitable material for microfluidics. Non-limiting examples of substrate material include silicon, glass, quartz, ceramics, or polymeric material, and combinations thereof, such as glass-epoxy laminates, glass/silicon wafers, etc.

As used herein, “magnet” refers to or includes a material to generate a magnetic field to trap magnetic particles in flow. Trapping refers to or includes capturing and immobilizing for a predetermined interval. The magnet includes conducting material, such as iron, nickel, cobalt, copper, gold, or aluminum, and mixtures or alloys thereof, or magnetically susceptible features to be magnetized with an external magnet.

As used herein, “sample fluid” refers to or includes a liquid composition for analysis. The sample fluid may be prepared by adding an immunoreactive magnetic particle to a fluid specimen.

As used herein, “immunoreactive magnetic particle” refers to and includes a magnetic particle coated with any capture antibody, or any antibody fragment, that may be used in a sandwich ELISA. The Immunoreactive magnetic particle includes a capture antibody. As used herein, “capture antibody” refers to or includes a paratope, or fragment thereof, also known as an antigen binding site to specifically bind a target antigen. Selection of a suitable capture antibody is based on the target antigen: a suitable capture antibody is capable of specific binding to a first epitope of the target antigen without interfering with the binding sight of the second antibody of the sandwich ELISA. The specificity of an immunoreactive magnetic particle of the present disclosure refers to the goodness of fit between capture antibody- or antibody fragment-combining site and the epitope. A variety of different methods of engineering, producing, and evaluating the relative binding capabilities of antibodies/antibody fragments to an analyte of interest, and conjugating the antibody or antibody fragment to a magnetic particle may be used. Specificity may be confirmed by any method or procedure for antibody validation.

The immunoreactive magnetic particle includes a material that responds to a magnetic field. The magnetic particle may be selected from nanoparticles and microparticles, such as nanospheres and microspheres. In some examples, the magnetic particle is a magnetic bead. Non-limiting examples of suitable magnetic particles include paramagnetic materials, superparamagnetic materials ferromagnetic materials, ferrimagnetic materials, and metamagnetic materials, such as iron, nickel, and cobalt, as well as metal oxides (e.g., Fe₃O₄, BaFe₁₂O₁₉, CoO, NiO, Mn₂O₃, Cr₂O₃, and CoMnP. In some examples, the surface of the magnetic particle is functionalized with a chemical moiety or functional group to permit attachment or conjugation of the capture antibody thereto. As used herein, “reporter fluid” refers to or includes a liquid composition including the immunoassay reporter, as described above.

In some examples, the droplet ejector comprises a thermal resistor. As used herein, “thermal resistor” refers to a heater that is capable of rapid, local superheating of a small volume of liquid (sample fluid, reporter fluid, etc.).

In some examples, the microfluidic network comprises a wash fluid channel in fluidic communication with the microfluidic channel upstream of the region, wherein the wash fluid channel comprises a constriction and a resistor to pump wash fluid through the constriction. As used herein, “wash fluid” refers to a buffer suitable for use with enzyme-linked immunocomplexes, such as buffered saline optionally including a detergent.

In some examples, the microfluidic network comprises a dilution fluid channel in fluidic communication with the microfluidic channel at a location proximate the droplet ejector. As used herein, “dilution fluid channel” refers to or includes a path through which a volume of reporter fluid may flow to the microfluidic channel and dilute the immunoreactive magnetic particles suspended therein.

In some examples, the substrate comprises a resistor to agitate immunoreactive magnetic particle in the region of the microfluidic channel adjacent to the magnet. As used herein, “a resistor to agitate” refers to or includes a fluid driver to selectively agitate fluid in the region and thereby agitate the immunoreactive magnetic particles to facilitate release from the magnetic field of the magnet. The resistor may include any device to move fluid within microfluidic channel, such as a micromixer, piezoelectric pump or a thermal micro-pump or a thin film resistor that is integrated with the substrate.

An example method for dispensing an immunoreactive magnetic particle and immunoassay reporter in a fluid droplet is also described. The example method comprises actuating a droplet ejector coupled to a distal end of a microfluidic network to draw a fluid specimen comprising a suspension of immunoreactive magnetic particles and a reporter enzyme conjugate into a microfluidic channel, wherein if a target antigen is present, the target antigen, an immunoreactive magnetic particle, and the reporter enzyme conjugate form an enzyme-linked immunocomplex in the fluid specimen; applying a magnetic field in a region of the microfluidic channel upstream of the droplet ejector to immobilize the immunoreactive magnetic particles on a surface of the microfluidic channel; flowing a fluid comprising a signal precursor over the immobilized immunoreactive magnetic particles, whereby non-immobilized components of the fluid specimen are removed from the region; removing the magnetic field to release the immunoreactive magnetic particles; and dispensing a fluid droplet from the droplet ejector, the fluid droplet comprising a portion of the released immunoreactive magnetic particles and the signal precursor. The term “actuating” refers to or includes an act that causes an operation of the droplet ejector that causes the specimen fluid to flow into the microfluidic network. The phrase “applying a magnetic field” refers to or includes using a magnetic field to immobilize the immunoreactive magnetic particles on a surface of the microfluidic channel, and/or put the magnetic field into operation or position for the stated purpose/effect.

In some examples, the method comprises monitoring the dispensed fluid droplet for a signal catalyzed by the enzyme-linked immunocomplex, wherein detection of the signal indicates the target antigen is present in the fluid specimen. Monitoring may comprise detecting a chromogenic, fluorescent or chemiluminescent signal produced by the enzyme-linked immunocomplex.

In some examples, the fluid droplet is dispensed into a chamber filled with an oil immiscible with the fluid droplet. As used herein, an oil that is immiscible is incapable of being mixed with or attaining homogeneity with the fluid droplet. As used herein, “chamber” refers to or includes an enclosed and/or semi-enclosed region of a receptacle.

In some examples, a plurality of droplets are dispensed in the oil in a planar array or a three dimensional array. As used herein, “array” refers to or includes ordered series or arrangement, such as in row and/or columns.

In some examples, the method includes partitioning the dispensed droplets. For example, the plurality of droplets dispensed as an array may include droplets from more than one sample or populations of droplets specific for different analytes, and partitioning may allow samples to be grouped together within the array. As used herein, “partitioning” refers to or includes dividing the dispensed droplets into discrete populations. Partitioning may comprise dispensing a separation fluid droplet in the oil adjacent to a droplet of the plurality. As used herein, “separator fluid” refers to a fluid to delineate a boundary between dispensed droplets of the plurality.

An example system for dispensing an immunoreactive magnetic particle and immunoassay reporter in a fluid droplet is also described. The example system comprises: an immunoassay droplet generator comprising: a microfluidic network comprising: a microfluidic channel to receive a sample fluid comprising a set of enzyme-linked immunocomplexes comprising an immunoreactive magnetic particle, the microfluidic channel comprising a region to release capture the immunoreactive magnetic particles in response to a magnetic field; a signal precursor fluid channel in fluidic communication with the microfluidic channel at a position fluidically upstream of the region to releasably capture the immunoreactive magnetic particles and to introduce a signal precursor to the enzyme-linked immunocomplexes; and an outlet coupled to a distal end of the microfluidic channel comprising a droplet ejector to dispense an immunoassay droplet; and, an oil-filled chamber positioned to receive the dispensed immunoassay droplet, wherein the oil is immiscible with the immunoassay droplet. As used herein, “signal precursor” refers to and/or includes an immunoassay reporter as described above.

In some examples, the system comprises a signal detector. As used herein, “signal detector” refers to or includes a device or instrument designed to detect the presence of the signal generated by the immunoassay reporter, and/or emit a signal in response. The signal detector may comprise an optical sensor, contact image sensor (CIS), photodiode, LED, laser diode, photoresistor, photomultiplier tube (PMT), charge coupled device (CCD), complementary metal oxide semiconductor (CMOS), mirror or beamsplitter, lens, rod lens array, self-focusing lens array, optical filter, colorimeter, spectrophotometer, photodetector, fluorescence imager, plate reader, fluorescent microscope, confocal fluorimeter, camera, scanner, light or illumination source, an excitation source, laser, digital image processing system, or a combination thereof. The signal detector may comprise a confocal point fluorimeter.

The present disclosure includes an example system for dispensing an immunoreactive magnetic particle and immunoassay reporter in a fluid droplet. The system includes a microfluidic channel supported by a substrate, a sample fluid region having a sample inlet in fluidic communication with the microfluidic channel, the sample inlet comprising a sample fluid driver, a reporter fluid inlet in fluidic communication with the microfluidic channel and comprising a reporter fluid driver; a magnet integrated with the substrate within a region of the microfluidic channel downstream from the sample fluid inlet and the reporter fluid inlet; a droplet ejector coupled to a distal end of the microfluidic channel; and a controller operatively coupled to the magnet, the fluid drivers, and the droplet ejector. The controller includes a processing unit and a non-transient computer-readable medium containing instructions that when executed, cause the processing unit to: actuate the sample driver to introduce a fluid specimen from the sample fluid region into the microfluidic channel via the sample inlet, the fluid specimen including a suspension of enzyme-linked immunocomplexes comprising an immunoreactive magnetic particle; activate a magnetic field in the region of the microfluidic channel to immobilize the immunoreactive magnetic particles on a surface of the microfluidic channel; actuate the reporter fluid driver to flow a reporter fluid over the immobilized immunoreactive magnetic particles, the reporter fluid comprising a signal precursor; deactivate the magnetic field to release the immunoreactive magnetic particles; and actuate the droplet ejector for dispensing a fluid droplet from the droplet ejector, the fluid droplet comprising a portion of the released immunoreactive magnetic particles and the signal precursor.

In some examples, the system includes a mixing resistor in the region. The mixing resistor may be operatively coupled to the controller and the instructions, when executed, may cause the processing unit to actuate the mixing resistor when the magnetic field is deactivated. The mixing resistor is actuated to resuspend the released immunoreactive magnetic particles in the microfluidic channel.

In some examples, the system includes a separation fluid inlet in fluidic communication with the microfluidic channel. The separation fluid inlet includes a separation fluid driver. The separation fluid driver is operably coupled to the controller and the instructions, when executed, cause the processing unit to: actuate the separation fluid driver to introduce a separation fluid to the microfluidic channel after a predetermined volume of the fluid specimen has been introduced to the microfluidic channel; and actuate the droplet ejector to dispense a droplet of separator fluid.

In some examples, the sample fluid region of the system includes a series of N sample inlets in fluidic communication with the microfluidic channel, wherein N is a positive integer. Each inlet is positioned fluidically downstream from the region and includes a multiplexing fluid driver operably coupled to the controller, wherein for an integer n=0 to N−1, the instructions, when executed, cause the processing unit to: actuate the multiplexing fluid driver of an nth sample inlet to introduce a predetermined volume of an nth sample fluid after the droplet of separator fluid has been dispensed, each N sample fluids including a suspension of enzyme-linked immunocomplexes bound to magnetic particles; activate the magnetic field to immobilize the magnetic particles of the nth sample fluid on the surface of the microfluidic channel; actuate the reporter fluid driver to introduce the reporter fluid over the immobilized magnetic particles; deactivate the magnetic field to release the magnetic particles; and actuate the droplet ejector to dispense a droplet comprising a portion of the enzyme-linked immunocomplexes bound to magnetic particles and the signal precursor.

In some examples, the system includes a dilution fluid inlet in fluidic communication with the microfluidic channel at a location proximate to the droplet ejector. The location is fluidically downstream from the region and includes a dilution fluid driver. The dilution fluid driver is operably coupled to the controller and the instructions, when executed, cause the processing unit to: actuate the dilution fluid driver to dilute the portion of the released immunoreactive magnetic particles; and actuate the droplet ejector to dispense a digital droplet from the droplet ejector, the digital droplet comprising a single immunoreactive magnetic particle and the signal precursor. As used herein, “digital droplet” refers to or includes a droplet that allows single particle and/or single molecule detection.

Turning to the accompanying drawings, devices 100, 200, 300, and 400, and systems 500, 600, 700, 1000, 1100, 2200, and 1300 described therein may have particular utility for personal and point-of-care clinical diagnostics by reducing the volume of sample and reagents compared to prior ELISA platforms.

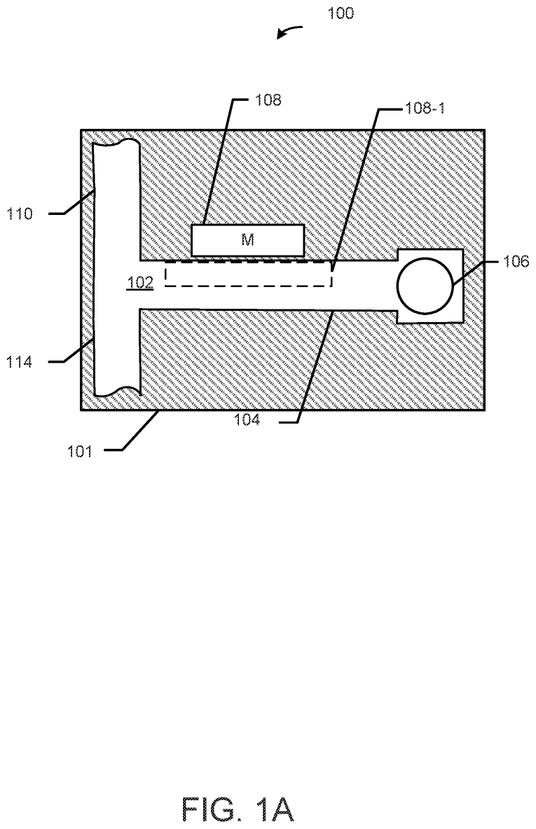

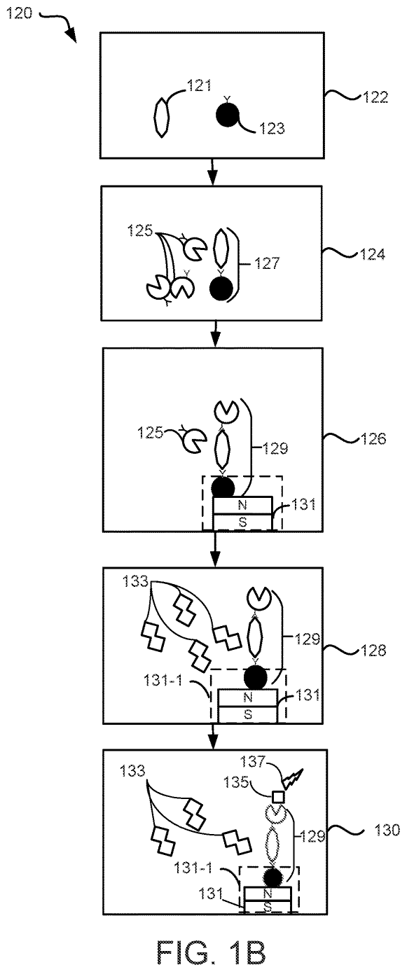

FIG. 1A illustrates an example device 100 for dispensing an immunoreactive magnetic particle and immunoassay reporter in a fluid droplet, consistent with the present disclosure. Device 100 may be used to receive and flow a sample fluid containing an immunoreactive magnetic particle therethrough, immobilize the immunoreactive magnetic particle, and introduce a reporter fluid to the immobilized immunoreactive magnetic particle. The sample fluid may be any liquid composition for analysis. The sample fluid may be prepared by adding an immunoreactive magnetic particle to a fluid specimen. As used herein, “immunoreactive magnetic particle” refers to and includes a magnetic particle coated with any capture antibody, or any antibody fragment, that may be used in a sandwich ELISA. As used herein, “reporter fluid” refers to or includes a liquid composition including a molecule for generating a detectable signal in the presence of an enzyme-linked immunocomplex. An enzyme-linked immunocomplex may include a complex formed between a target antigen and an enzyme-conjugated antibody, or antibody fragment, having an affinity to bind the target antigen. An antigen may be any molecule or structure thereof, or particulate matter, capable of specifically binding to the conjugated antibody or fragment thereof. Detection of the generated signal indicates the fluid specimen contains the antigen.

Device 100 includes a microfluidic network 102 supported by a substrate 101. In some examples, microfluidic network 102 may transport various volumes of fluid, such as microliter (μL), nanoliter, picoliter, or femtoliter volumes, through the microfluidic device. Microfluidic network 102 may be formed of an etched, microfabricated, and/or micromachined portion of substrate 101. Substrate 101 may be formed of any suitable material for microfluidics. Non-limiting examples of substrate material include silicon, glass, quartz, ceramics, or polymeric material, and combinations thereof, such as glass-epoxy laminates, glass/silicon wafers, etc.

In some examples, microfluidic network 102 includes a microfluidic channel 104, a sample fluid channel 110 and a reporter fluid channel 114. In some examples, microfluidic channel 104 includes sample fluid channel 110 to deliver a sample fluid to the microfluidic channel 104. Reporter fluid channel 114 may be positioned proximate the sample fluid channel 110 and to deliver a fluid comprising an immunoassay reporter to the microfluidic channel. Although a specific arrangement of microfluidic network 102 is shown in FIG. 1A, devices of the present disclosure may have other arrangements in other examples.

In some examples, a magnet 108 is integrated with substrate 101 within a region 108-1 of the microfluidic channel downstream from sample fluid channel 110 and reporter fluid channel 114. As used herein with respect to magnet 108, “integrated” refers to and includes a combination of the substrate and the magnet to provide a functional substrate for binding and/or manipulation of the immunoreactive magnetic particle using magnetic forces. Manipulation may include transporting the immunoreactive magnetic particle by pulling it from the sample fluid channel towards the region. Selective activation of the magnet during fluid flow facilitates removal of unbound components of the sample fluid from the immunoreactive magnetic particle. In some examples, magnet 108 is positioned adjacent to a wall of microfluidic channel 104 for releasably immobilizing the immunoreactive magnetic particle in the region of the microfluidic channel. Although illustrated positioned adjacent to a side wall, in some examples, magnet 108 may be positioned at the bottom of microfluidic channel 104.

Magnet 108 may include a material to generate a magnetic field to trap magnetic particles in flow. As used herein “trap” refers to or includes the capture and immobilization for a predetermined time interval. In some examples, the magnet 108 may include conducting material, such as iron, nickel, cobalt, copper, gold, or aluminum, and mixtures or alloys thereof, or magnetically susceptible features to be magnetized with an external magnet. For example, a magnet may include neodymium iron boron (NdFeB) alloy or may include nickel to be magnetized with external NdFeB magnets. In some examples, magnet 108 may include an electromagnet. Depending on the configuration and flow rate of the sample fluid, a current for trapping the immunoreactive magnetic particle may be within a range of 500 mA to 30 mA. In some examples, magnet 108 may be a micro-fabricated magnet, such as a micro-fabricated electromagnet. Non-limiting methods of microfabrication include sputtering, electroplating, evaporation, vapor deposition, etching and lift-off techniques. In some examples, the region 108-1 may include a plurality of magnet 108. For example, the region 108-1 may include a pair of 3D electromagnets integrated with poles adjacent to microfluidic channel 104. In some examples, the magnet 108 may have a planar form or may include a plurality of overlapping planar magnets. A planar magnet may be linear or curvilinear, such as a wire. The wire may be integrated in serpentine and/or spiral shapes. In some examples, magnet 108 may include a connection to an external power supply. In some examples, magnet 108 may include electrical insulation, such as a polymeric and/or ceramic layer. In some examples, magnet 108 may include a thermal insulation to maintain the temperature of the fluid in the region in the presence of Joule heating.

In the example of FIG. 1A, droplet ejector 106 is coupled to an end of microfluidic channel 104 distal to fluid channels 110 and 114 for dispensing a fluid droplet from the device. Droplet ejector 106 may include a piezoelectric device or a thermal resistor to eject a droplet of fluid. In some examples, a thermal resistor may rapidly, superheat a small volume of liquid (sample fluid, reporter fluid, etc.) proximate the thermal resistor. In some examples, the droplet ejector 106 may include an open nozzle (not shown) permitting a vapor bubble formed by the thermal resistor to eject a droplet of fluid into the ambient environment of the device 100. In some examples, the droplet ejector 106 may dispense the droplet at a specific location. In some examples, the droplet ejector 106 may be capable of partitioning the fluid in the microfluidic channel to release a discrete number of immunoreactive magnetic particles (e.g., 0, 1, 2, 3, etc.) in the fluid droplet. In some examples, the droplet ejector 106 may be tuned to release a desired number of immunoreactive magnetic particles per droplet.

FIG. 1B is a flow diagram of an example method for dispensing an immunoreactive magnetic particle and immunoassay reporter in a fluid droplet. In some examples, the device 100 of FIG. 1A may be used to implement the method 120 of FIG. 1B. At 122, method 120 includes preparing a sample fluid by contacting an analyte 121 with an immunoreactive magnetic particle 123. The sample fluid may be prepared by mixing an immunoreactive magnetic particle 123 and a fluid specimen containing analyte 121, in any order. As used herein, “fluid specimen” refers to or includes a sample for testing. A fluid specimen may be or include a biological specimen, although examples are not limited to biological specimens. The term “biological specimen” refers to, for example, whole blood, lymphatic fluid, serum, plasma, sweat, tear, saliva, sputum, cerebrospinal (CSF) fluids, amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs or washes (e.g., oral, nasopharangeal, optic, rectal, intestinal, vaginal, epidermal, etc.). The fluid specimen may include a diluent. The diluent may be used to dilute a liquid fluid specimen or suspend a solid or semi-solid specimen. The composition of the diluent may vary based on the specimen being investigated. The diluent may be a pH-balanced salt solution, such as a buffered saline. In some cases, the diluent includes detergent and/or blocking agent selected to achieve the desired or needed sensitivity and optionally to reduce background and/or non-specific binding of immunoassay reagents, such as the immunoreactive magnetic particle. The fluid specimen may include phosphate- or TRIS-buffered saline, and optionally surfactant and/or albumin to bind to all potential sites of nonspecific interaction on the analyte.

Immunoreactive magnetic particle 123 includes a capture antibody. As discussed above, the capture antibody may include a paratope, or fragment thereof, as an antigen binding site for specifically binding a target antigen. Selection of a suitable capture antibody is based on the target antigen: a suitable capture antibody is capable of specific binding to a first epitope of the target antigen without interfering with the binding sight of the second antibody of the sandwich ELISA. The specificity of an immunoreactive magnetic particle of the present disclosure refers to the goodness of fit between capture antibody- or antibody fragment-combining site and the epitope. Various different methods of engineering, producing, and evaluating the relative binding capabilities of antibodies/antibody fragments to an analyte of interest, and conjugating the antibody or antibody fragment to a magnetic particle may be used. Specificity may be confirmed by any method or procedure for antibody validation. Although immunoreactive magnetic particle 123 is illustrated with a single capture antibody thereon, an immunoreactive magnetic particle may include a plurality of capture antibodies. The surface of the particle facilitates high density binding of capture antibodies without sterically hindering the capture of multiple molecules of target analytes by a single particle.

Immunoreactive magnetic particle 123 includes a magnetically responsive material, i.e., a material that responds to a magnetic field. The magnetic particle may be selected from nanoparticles and microparticles, such as nanospheres and microspheres. In some cases, the magnetic particle is a magnetic bead. Non-limiting examples of suitable magnetic particles include paramagnetic materials, superparamagnetic materials ferromagnetic materials, ferrimagnetic materials, and metamagnetic materials, such as iron, nickel, and cobalt, as well as metal oxides (e.g., Fe₃O₄, BaFe₁₂O₁₉, CoO, NiO, Mn₂O₃, Cr₂O₃, and CoMnP. In some examples, the surface of the magnetic particle is functionalized with a chemical moiety or functional group to permit attachment or conjugation of the capture antibody thereto.

Returning to FIG. 1B, at 124, after incubating, and optionally washing the analyte-bound immunoreactive magnetic particle 127, an enzyme-conjugated antibody 125 is added to the mixture. The antibody conjugated to the enzyme of the enzyme-conjugated antibody 125 is immunoreactive with a second epitope of the target antigen. In the case that a specimen fluid does not include an analyte of interest, the enzyme-conjugated antibody 125 may not form a complex with the analyte-bound immunoreactive magnetic particle 127. The prepared sample fluid may be introduced to device 100 via sample fluid channel 110, for example.

At 126, the sample fluid including enzyme-linked immunocomplex 129 and unbound enzyme-conjugated antibody 125 is flowed to a region 131-1 of a microfluidic channel adjacent to a magnet 131. Activation of magnet 131 releasably immobilizes the enzyme-linked immunocomplex 129 within the region 131-1. Magnet 131 is illustrated as being positioned on the bottom of the microfluidic channel; however, other arrangements are contemplated, including any one of the arrangements described for magnet 108.

At 128, a reporter fluid that includes immunoassay reporter 133 is flowed into the region 131-1 of the microfluidic channel with enzyme-linked immunocomplex 129. Unbound or non-specifically bound substances that may be present in the sample fluid, such as enzyme-conjugated antibody 125, are washed away from the magnetically immobilized enzyme-linked immunocomplex 129 to reduce background signal related to unbound or non-specifically bound conjugated antibody (e.g., 125). In some examples, the reporter fluid flows until all of the sample fluid flows out of the microfluidic network. For example, the reporter fluid flow may continue until reporter fluid is detected by a sensor (not shown), flowing out of the microfluidic network, such as into a waste fluid receptacle. The sensor may be integrated with the microfluidic network or positioned proximate the microfluidic network. For example, the microfluidic network may include an optically transparent window to permit detection of the reporter fluid by an optical sensor at a specific location. In other examples, the sensor may detect a change in conductivity or temperature associated with the removal of components of the fluid sample that may contribute to background signal and/or introduction of the reporter fluid. In some examples, the reporter fluid flows for a predetermined duration. In some examples, the reporter fluid flow is intermittent, such that the magnetically immobilized enzyme-linked immunocomplex is contacted multiple times with reporter fluid. In some examples, the flow rate of reporter fluid is adjusted for volume, duration, and/or agitation within the microfluidic channel to separate unbound or non-specifically bound substances within the region 131-1 while maintaining the specific bonds of the immobilized enzyme-linked immunocomplex 129 to improve immunoassay accuracy.

The immunoassay reporter 133 may be a photogenic molecule, such as a fluorogenic, colorometric, spectrophotogenic, and/or chemiluminescent substrate of the conjugated enzyme. For example, the photogenic molecule may be a fluorogenic substrate of β-galactosidase such as 4-Methylumbelliferyl-β-D-Galactopyranside (MUG), Carboxyumbelliferyl β-D-Galactopyranoside (CUG), Fluorescein di-β-D-galactopyranoside; Spiro(isobenzofuran-1(3H),9-(9H) xanthen)-3one, 3,6-bis(β-D-galactopyranosyloxy) (FDG), Dichlorofluorescein di-Galactoside (DCFDG), Fluorescein mono-β-D-galactopyranoside (FMG), 4-Trifluoromethylumbelliferyl-β-D-Galactopyranoside (TMFU-Gal), Resorufin β-D-Galactopyranoside (Res-Gal), and DDAO β-D-Galactopyranoside (DDAO-Gal), or a fluorogenic substrate of horseradish peroxidase, such as the photogenic molecules sold under the trade names QuantaBlu Fluorogenic Substrate, QuantaRed Enhanced Chemifluorescent Substrate, Amplex Red Reagent and Amplex UltraRed Reagent. In some cases, the photogenic molecule may be a colorogenic substrate of horseradish peroxidase (HRP) or alkaline phosphatase (AP) such as 3,3′,5,5′-tetramethylbenzidine (TMB), 2,2′-Azinobis [3-ethylbenzothiazoline-6-sulfonic acid]-diammonium salt (ABTS), o-phenylenediamine dihydrochloride (OPD), p-Nitrophenyl Phosphate, Disodium Salt (PNPP). In some cases, the photogenic molecule may be a colorogenic substrate of a beta-galactoside, horseradish peroxidase, or alkaline phosphatase (AP), such as ortho-Nitrophenyl-beta-galactoside (ONPG), or the photogenic molecules sold under the trade names CSPD, CDP-Star, DynaLight Substrate, ELISA Pico Chemiluminescent Substrate, and SuperSignal ELISA Femto Maximum Sensitivity Substrate.

At 130, an immunoassay reporter 133 is converted to a signal molecule 135 to produce a signal 137. The signal is generated by the activity of the enzyme of enzyme-linked immunocomplex 129. In the case that a specimen fluid does not include an analyte of interest, the enzyme is not present in the fluid and no signal may be produced.

After introduction of the immunoassay reporter 133, the enzyme-linked immunocomplex 129 is released from magnet 131 by removing the magnetic field. After release, enzyme-linked immunocomplex 129 and immunoassay reporter 133 are dispensed in a fluid droplet via a droplet ejector coupled to a distal end of the microfluidic channel. The fluid droplet may be monitored for signal 137 using any suitable means for detecting the signal, such as an optics system or monitoring assembly. Signal 137 may be a change in a radiant quality of the assay, such as a change in the color, or emission of light (e.g., fluorescence or chemiluminescence). Analysis of the results may be qualitative and/or quantitative.

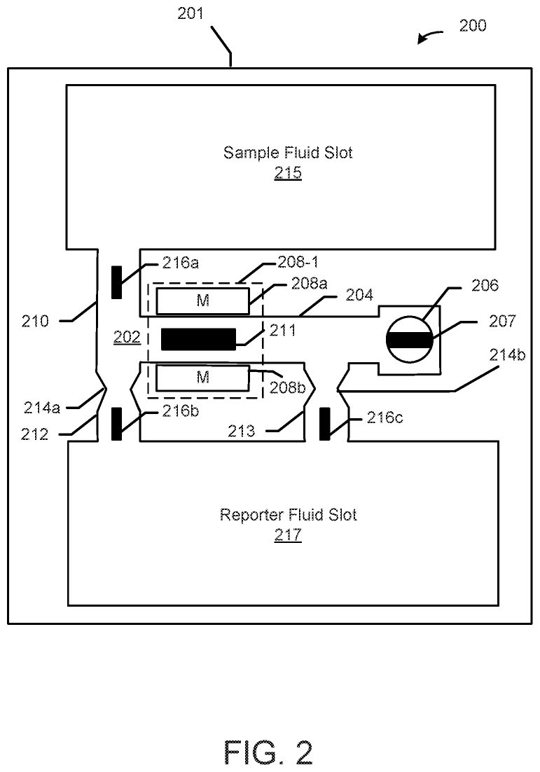

FIG. 2 illustrates an example device 200 for dispensing an immunoreactive magnetic particle and immunoassay reporter in a fluid droplet, consistent with the present disclosure. Device 200 may be used for performing an ELISA as described above.

In the example of FIG. 2, device 200 includes a microfluidic network 202 supported by a substrate 201. Substrate 201 may be similar to the substrate of device 100. Microfluidic network 202 may be formed of an etched, microfabricated, and/or micromachined portion of substrate 201. Microfluidic network 202 includes a microfluidic channel 204, a sample fluid channel 210, a reporter fluid channel 212, and a dilution fluid channel 213. Sample fluid channel 210 includes fluid driver 216a to deliver a sample fluid from a sample fluid feed slot 215 to the microfluidic channel 204. Reporter fluid channel 212 includes a reporter fluid driver 216b to deliver a fluid comprising an immunoassay reporter from reporter fluid feed slot 217 to microfluidic channel 204. Reporter fluid channel 212 includes a constriction 214a to prevent fluid from sample fluid feed slot 215 and/or microfluidic channel 204 from flowing into reporter fluid feed slot 217. Dilution fluid channel 213 includes a dilution fluid driver 216c to deliver a dilution fluid to microfluidic channel 204 and includes a constriction 214b to prevent fluid from microfluidic channel 204 flowing into reporter fluid feed slot 217.

As used herein, the term “constriction” refers to a narrowing in at least one dimension (e.g., a decrease in diameter) of a channel. A constriction may be formed by one side of a channel having a protuberance projecting towards the other side of the channel, both sides of a channel having at least one protuberance projecting towards the other side of the channel, wherein such multiple protuberances are either aligned with one another or are staggered along the channel. Suitable constrictions include, for example, capillary break valves formed by sloped or tapered walls of the channel proving a decrease in diameter sufficient to stop capillary action. Constriction 214a and 214b may include similar or different structural features capable of restricting fluid flow from microfluidic channel 204 into channels fluidically connected thereto.

Fluid drivers 216a, 216b and 216c may be any device used to selectively move and discharge fluid from a fluid feed slot to microfluidic channel 204. In some example, fluid drivers 216a, 216b and 216c may include resistors, such as those previously described in connection with FIG. 1A. In some examples, fluid drivers 216a, 216b and 216c include a piezoelectric pump or a thermal micro-pump. The thermal micro-pump may include a thin film resistor integrated with the substrate. Fluid drivers 216a, 216b and 216c may be the same or different.

As illustrated in FIG. 2, dilution fluid channel 213 is in fluidic communication with reporter fluid feed slot 217. Thus, the dilution fluid channel may deliver a volume of reporter fluid to microfluidic channel 204. Fluid feed slot 215 and/or 217 may be machined into the substrate. Fluid feed slots 215 and/or 217 may accommodate a large volume of fluid.

In some examples, magnets 208a and 208b are integrated with substrate 201 within a region 208-1 of microfluidic channel 204 downstream from sample fluid channel 210 and reporter fluid channel 212. Magnets 208a and 208b may be positioned adjacent to a wall of microfluidic channel 204 for releasably immobilizing the immunoreactive magnetic particle in the region 208-1. One or both magnets 208a and 208b may be similar to magnet 108 above. The region further includes a fluid driver 211 to selectively agitate immunoreactive magnetic particles released from the region by deactivation of a magnetic field. Fluid driver 211 may include any device that moves fluid within microfluidic channel 204, such as a resistor, micromixer, piezoelectric pump or a thermal micro-pump. The thermal micro-pump may include a thin film resistor integrated with the substrate.

A droplet ejector 206 is coupled to a distal end of microfluidic channel 204, as previously described. In some examples, droplet ejector 206 includes a thermal resistor 207 to eject a droplet of fluid. The droplet ejector may include an open nozzle permitting a vapor bubble formed by thermal resistor 207 to eject a droplet of fluid into the ambient environment of the device. Droplet ejector 206 may dispense the droplet at a specific location and/or be tuned to release a desired number of immunoreactive magnetic particles per droplet, such as a single magnetic particle per droplet for precise quantitative measurement of an analyte present in the sample fluid.

Although a specific arrangement of microfluidic network 202 is shown in FIG. 2, devices of the present disclosure may have other arrangements in other examples.

Introduction of the reporter fluid via dilution fluid channel 213 may facilitate dilution of immunoreactive magnetic particles that have been released from the magnetic field to achieve a digital droplet ELISA, conferring additional utility for device 200 for personal and point-of-care clinical diagnostics. The dilution may permit a digital droplet ELISA whereby a single immunoreactive magnetic particle may be dispensed in a droplet of reporter fluid.

Where a sample fluid includes a target antigen, the dispensed immunoreactive magnetic particle includes an enzyme-conjugated antibody, such as enzyme conjugated antibody 125 described above. The enzyme reacts with molecules of the immunoassay reporter to produce a signal. Confined within a droplet of nano-, pico-, femto-, or attoliter volume, the signal generated by the enzyme may achieve detectable concentration faster and using less reagent than plate-based immunoassays, even where a single target antigen is bound to the immunoreactive magnetic particle. Digital droplet ELISA may facilitate counting of the number of dispensed droplets containing signal, and quantification of a target antigen present at low or very low concentration within the sample fluid.

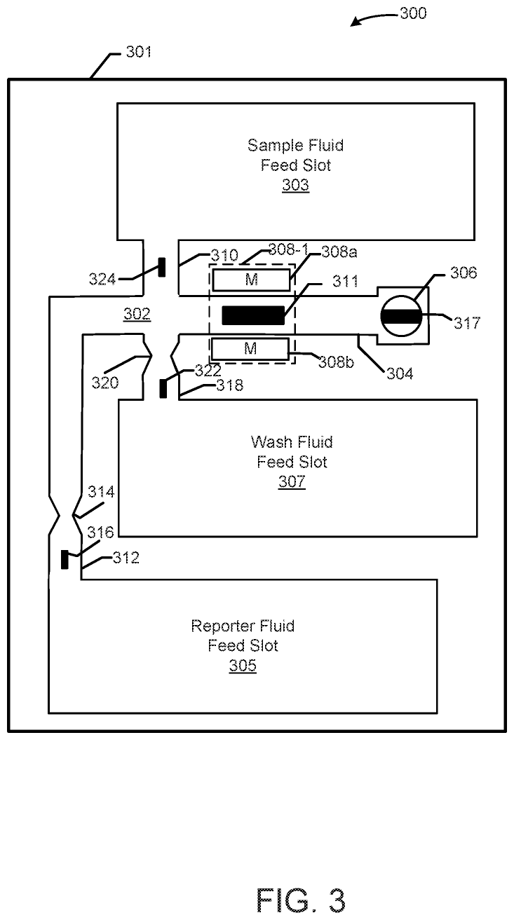

FIG. 3 illustrates another example device for dispensing an immunoreactive magnetic particle and immunoassay reporter in a fluid droplet, consistent with the present disclosure. Device 300 may be used for performing an ELISA as described above. In the example of FIG. 3, device 300 includes a microfluidic network 302 supported by a substrate 301. Substrate 301 may be similar to the substrate of device 100 and/or 200. Microfluidic network 302 may be formed of an etched, microfabricated, and/or micromachined portion of substrate 301.

In some examples, microfluidic network 302 includes a microfluidic channel 304, a sample fluid channel 310, a reporter fluid channel 312, and a wash fluid channel 318. Sample fluid channel 310 may include fluid driver 324 to deliver a sample fluid from a sample fluid feed slot 303 to the microfluidic channel 304. Reporter fluid channel 312 may include a reporter fluid driver 316 to deliver a fluid comprising an immunoassay reporter from reporter fluid feed slot 305 to microfluidic channel 304. Reporter fluid channel 312 may include a constriction 314 to prevent fluid from sample fluid feed slot 303 and/or microfluidic channel 304 from flowing into reporter fluid feed slot 305. Wash fluid channel 318 includes a wash fluid driver 322 to deliver a wash fluid to microfluidic channel 304 from wash fluid feed slot 307. In some examples, the wash fluid includes a buffer suitable for use with enzyme-linked immunocomplexes, such as buffered saline optionally including a detergent. The wash fluid is formulated to remove unbound or non-specifically bound components of the sample fluid. The wash fluid may stabilize specific interactions in the immunocomplex and/or stabilize the conjugated reporter enzyme. In various examples, the wash fluid feed slot may contain a wash fluid containing phosphate buffered saline (PBS) with 0.05-0.1% Tween-20. Tris-buffered saline may be used in some examples. Wash fluid channel 318 includes a constriction 320 to prevent fluid in microfluidic channel 304 flowing into wash fluid feed slot 307. Fluid feed slot 303, 305, or 307 may be machined into the substrate. Fluid feed slot 303, 305, and/or 307 may accommodate a large volume of fluid. Constrictions 314 and 320 may include similar or different structural features capable of restricting fluid flow from microfluidic channel 304 into channels fluidically connected thereto.

Fluid drivers 316, 322, and 324 may be independently selected from devices to selectively move and discharge fluid from a fluid feed slot to microfluidic channel 304. The fluid drivers 316, 322, and 324 may include resistors, such as those previously described in connection with FIG. 1A.

In some examples fluid drivers 316, 322, and 324 may be the same or different. Fluid drivers 316, 322, and 324 are independently activatable. For example, sample fluid driver 324 may be actuated to move a volume of sample fluid into microfluidic channel 304, then wash fluid driver 322 may be actuated to move a volume of sample fluid into microfluidic channel 304, and then reporter fluid driver 316 may be actuated to move a volume of reporter fluid into microfluidic channel 304. Each volume may be independently controlled. In some examples, fluid drivers 316, 322, and 324 include a piezoelectric pump or a thermal micro-pump. The thermal micro-pump may include a thin film resistor integrated with the substrate.

In some examples, magnets 308a and 308b are integrated with substrate 301 within a region 308-1 of microfluidic channel 304 downstream from fluid channels 310, 312, and 318. In some examples, magnets 308a and 308b are positioned adjacent to a wall of microfluidic channel 304 to releasably immobilize the immunoreactive magnetic particle in the region 308-1. One or both magnets 308a and 308b may be similar to magnet 108 as described above. The region further includes a fluid driver 311 to selectively agitate immunoreactive magnetic particles released from the region 308-1 by deactivation of a magnetic field. Fluid driver 311 may disperse the immunoreactive magnetic particles within the microfluidic channel to assist in dilution. Fluid driver 311 may include any device for moving fluid within microfluidic channel 304, such as a resistor, micromixer, piezoelectric pump or a thermal micro-pump. The thermal micro-pump may include a thin film resistor integrated with the substrate.

A droplet ejector 306 is coupled to a distal end of microfluidic channel 304. In some examples, droplet ejector 306 includes a thermal resistor 317 to eject a droplet of fluid. The droplet ejector may include an open nozzle permitting a vapor bubble formed by thermal resistor 317 to eject a droplet of fluid into the ambient environment of the device. Droplet ejector 306 may dispense the droplet at a specific location and/or be tuned to release a desired number of immunoreactive magnetic particles per droplet, such as a single magnetic particle per droplet for precise quantitative measurement of an analyte present in the sample fluid.

Although a specific arrangement of microfluidic network 302 is shown in FIG. 3, devices of the present disclosure may have other arrangements in other examples.

The wash fluid slot 307 and channel 318 may permit further reduction of the volume of reporter fluid and/or immunoassay reporter introduced to the microfluidic network as compared with devices 100, 200, or 300.

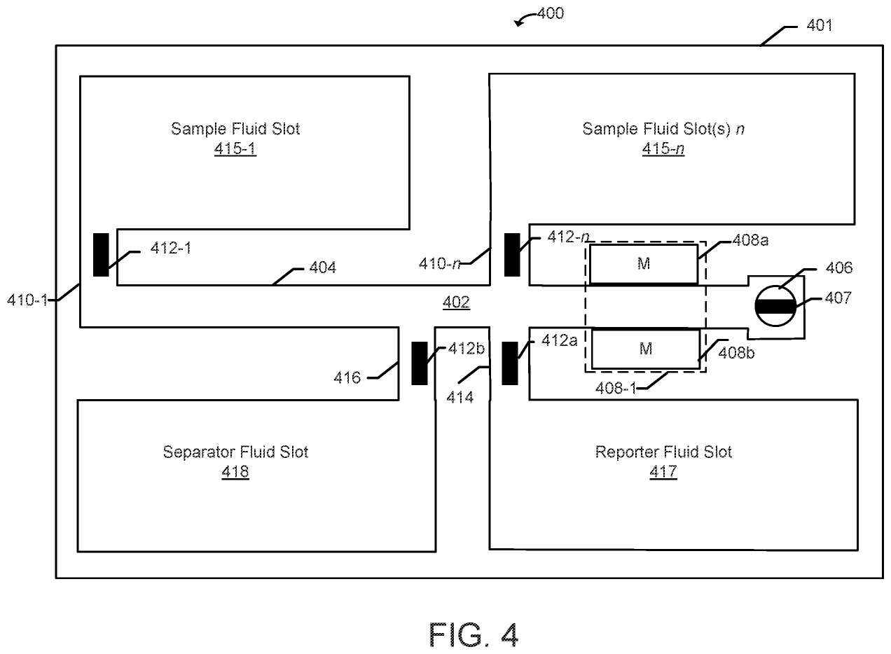

FIG. 4 illustrates another example device 400 for dispensing an immunoreactive magnetic particle and immunoassay reporter in a fluid droplet, consistent with the present disclosure. Device 400 may be used for performing an ELISA as described above.

In the example of FIG. 4, device 400 includes a microfluidic network 402 supported by a substrate 401. Substrate 401 may be similar to the substrate of device 100, 200 and/or 300. Microfluidic network 402 may be formed of an etched, microfabricated, and/or micromachined portion of substrate 401.

Microfluidic network 402 includes a microfluidic channel 404, a first sample fluid channel 410-1, an nth sample fluid channel 410-n, a reporter fluid channel 414, and a separator fluid channel 416, where n is a positive integer greater than 1. In some examples, microfluidic network 402 includes 5, 6, 7, 8, 9, 10, or more sample fluid channels. Sample fluid channels 410-1 thru 410-n include fluid drivers 412-1 thru 412-n, used to independently deliver a sample fluid from each of sample fluid feed slots 415-1 thru 415-n.

Reporter fluid channel 414 includes a reporter fluid driver 412a to deliver a fluid comprising an immunoassay reporter from reporter fluid feed slot 417 to microfluidic channel 404. Separation fluid channel 416 includes a separator fluid driver 412b to deliver a separator fluid to microfluidic channel 404 from separator fluid feed slot 418. In some examples, the separator fluid includes a fluid to provide a visual boundary between a droplet prepared from sample fluid feed slot 415-1 and a droplet prepared from sample fluid feed slot 415-n. Fluid feed slots 415-1 thru 415-n may each contain a reporter enzyme conjugated to a different target antigen, such as for a biomarker panel, may contain a reporter enzyme conjugated to a single target analyte, but each sample may be from a different subject, for example. A separator fluid may be a dye or ink, such as a dye or ink having an excitation and/or emission spectra that is distinguishable from a signal generated by the immunoassay reporter. Fluid feed slots 415-1 thru 415-n, 417, and 418 may be machined into the substrate, and/or accommodate a large volume of fluid.

In some examples, fluid drivers 412-1 thru 412-n, 412a, and 412b may be independently selected from devices to selectively move and discharge fluid from a fluid feed slot to microfluidic channel 404. The fluid drivers 412-1 thru 412-n, 412a, and 412b may include resistors, such as those previously described in connection with FIG. 1A. Fluid drivers 412-1 thru 412-n, 412a, and 412b may be the same or different. In some examples, fluid drivers 412-1 thru 412-n, 412a, and 412b are independently activatable. For example, sample fluid driver 412-1 may be actuated to move a volume of a first sample fluid into microfluidic channel 404, then reporter fluid driver 412a may be actuated to move a volume of reporter fluid into microfluidic channel 404, then separator fluid driver 412b may be actuated to move a volume of separator fluid into microfluidic channel 404, and then sample fluid driver 412-n may be actuated to move a volume of an nth sample fluid. Each volume may be independently controlled. In some examples, fluid drivers 412-1 thru 412-n, 412a, and 412b include a piezoelectric pump or a thermal micro-pump. The thermal micro-pump may include a thin film resistor integrated with the substrate.

In some examples, magnets 408a and 408b are integrated with substrate 401 within a region 408-1 of microfluidic channel 404 fluidically upstream of a droplet ejector 406, and positioned adjacent to a wall of microfluidic channel 404 to releasably immobilize the immunoreactive magnetic particle in the region 408-1. One or both magnets 408a and 408b may be similar to magnet 108 as described above.

Droplet ejector 406 is coupled to a distal end of microfluidic channel 404. In some examples, droplet ejector 406 includes a thermal resistor 407 to eject a droplet of fluid. The droplet ejector may include an open nozzle permitting a vapor bubble formed by thermal resistor 407 to eject a droplet of fluid into the ambient environment of the device. Droplet ejector 406 may dispense the droplet at a specific location and/or be tuned to release a desired number of immunoreactive magnetic particles per droplet, such as a single magnetic particle per droplet for precise quantitative measurement of an analyte present in the sample fluid.

Although a specific arrangement of microfluidic network 402 is shown in FIG. 4, devices of the present disclosure may have other arrangements in other examples. For example, microfluidic network may include constrictions and/or a fluid driver for agitating immunoreactive magnetic particles within microfluidic channel 404, as described for device 200 and/or 300, above.

The separator fluid channel 416 and sample fluid feed slots 415-1 and 415-n of device 400 may improve the efficiency of multiplexed ELISA as compared with prior multiplexed ELISA platforms.

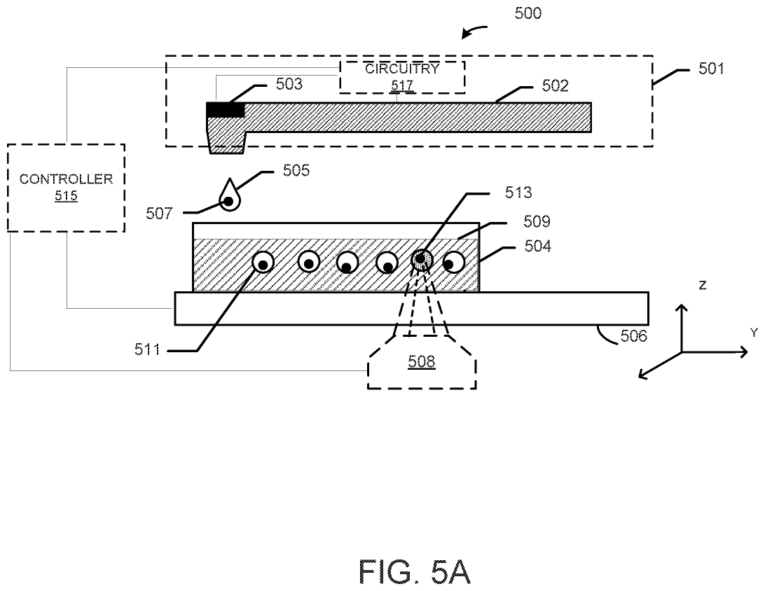

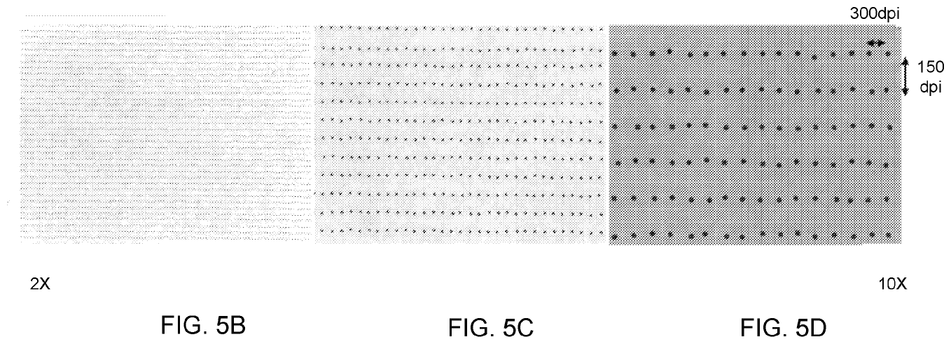

FIG. 5A illustrates an example system 500 for dispensing an immunoreactive magnetic particle and immunoassay reporter in a fluid droplet. System 500 may be used to perform a sandwich ELISA, consistent with the present disclosure. In the example of FIG. 5A, system 500 includes an immunoassay droplet generator 502, chamber 504 to contain an oil 509 and receive a dispensed droplet 505, sample platform 506 and optics system 508.

In some examples, system 500 may include optics system 508. Optics system 508 may include a device or component for gathering quantitative, qualitative, or semi-quantitative data. Optics system 508 may include a scanner. In some examples, optics system 508 may include a device or component for stage scanning signal detection, where chamber 504 is moved relative to optics system 508 or beam scanning signal detection, where 504 remains stationary relative to the device or component. In some examples, optics system 508 may include, for example, an optical sensor, contact image sensor (CIS), photodiode, LED, laser diode, photoresistor, photomultiplier tube (PMT), charge coupled device (CCD), complementary metal oxide semiconductor (CMOS), mirror or beamsplitter, lens, rod lens array, self-focusing lens array, optical filter, colorimeter, spectrophotometer, photodetector, fluorescence imager, plate reader, fluorescent microscope, confocal fluorimeter, camera, light or illumination source, such as an excitation source, laser, digital image processing system, or a combination thereof. In some cases, optics system 508 may include a device or component having a sensitivity to detect a signal generated by a single enzyme-linked immunocomplex.

In some examples, immunoassay droplet generator 502 may include elements of or be similar to device 100, 200, 300, or 400. In some examples, immunoassay droplet generator 502 may form part of an immunoassay dispensing instrument 501. For example, immunoassay droplet generator 502 may be inserted into immunoassay dispensing instrument 501. Immunoassay dispensing instrument 501 may be any instrument to drive or other allow the operation of immunoassay droplet generator 502, or component thereof, such as, printer, a mobile device, multimedia device, a secure microprocessor, a notebook computer, a desktop computer, an all-in-one system, a server, a network device, a controller, or a wireless device.

In some examples, immunoassay droplet generator 502 may include droplet ejector 503. In some examples, droplet ejector 503 dispenses a fluid droplet 505 having an immunoreactive magnetic particle 507 and an immunoassay reporter (not shown) into an oil-filled chamber 504. Mixing and mass transfer may be enhanced within a droplet, and a signal generated by an enzyme conjugated to the immunoreactive magnetic particle in the droplet, as described above, may produce a detectable signal more rapidly than prior ELISA platforms. In some examples, chamber 504 may be at least partially filled with any type of oil. In some examples, oil 509 is immiscible with the dispensed droplet and/or has a density lower than the fluid of the dispensed droplet. As a static, spatially-defined array may facilitate indexing and monitoring of immunoassay droplets over time, in some examples, the oil has a viscosity that limits or restricts movement of the dispensed droplets within chamber 504.

Oil 509 includes a row of dispensed immunoassay droplets 505, however, other patterns may be achieved. Positive droplet 513 includes a generated signal to confirm the presence of the target antigen. As illustrated, droplet 511 is not generating a signal and is, thus, negative for the target antigen. In some examples, oil 509 may include a silicone oil, hydrocarbon oil, fluorinated oil, or combination thereof. For example, the oil may include a polydimethylsiloxane (PDMS) such as DC200, hexa-methyl di-siloxane, or polyphenyl methylsiloxane such as AR200, dexadecan, tetraocta/dodecane, mineral oil, isoparaffin, vegetable oil, polymeric oils with fluorine-containing monomers and/or a perfluoropolyether group, such as FH/PFC/PFD/PFPH, HFE, FC40, FC70/FC77, or FC3283, and combinations thereof. In some examples, oil 509 may contain agent(s) to prevent coalescence of dispensed droplets. In some examples, the oil-filled chamber 504 includes a surfactant to stabilize the encapsulated droplet at the droplet-oil interface. The surfactant may be a water-in-oil emulsifier based on silicone, perfluorinated compounds, or organic molecules. The surfactant may depend on the composition of the droplet and/or oil 509. Non-limiting examples of surfactants include polyethylene glycol (PEG) tert-octylphenyl ether, sodium dodecylsulfate, cetyl PEG/polypropylene glycol (PPG) 10/1 dimethicone, fatty acid sorbitan esters, ethoxylated polysorbates, fatty acids, monolein, phospholipid, fatty alcohols, fluorinated-surfactants, and combinations thereof.

Chamber 504 may be any vessel capable of containing a volume of oil 509 to receive the dispensed droplets. In some examples, chamber 504 may include compartments, such as wells. In some examples, chamber 504 may have a cover or be otherwise sealable. Example chambers include, without limitation, 1-, 4-, and 8-well rectangular dishes, microwell plates, such as a 6-, 12-, 24-, 48-, 96-, 384-, or 1536-well plate, and a petri dish, such as a 50 mm, 100 mm, or a 300 mm petri dish. Chamber 504 may be constructed from any material that is compatible with the oil and/or optics system 508. In some examples, chamber 504 may include a waveguide chip for total internal reflection fluorescence (TIRF) acquisition of a wide-field full image, or a super-resolution modality such as single molecule localization microscopy (SMLM), entropy based super-resolution imaging (ESI), or structured illumination microscopy (SIM).

As illustrated, sample platform 506 supports chamber 504. In some examples, sample platform 506 is fixed, and droplet generator 502 is capable of movement relative to sample platform 506. In some examples, sample platform 506 includes a movable stage, such as a motorized stage. The stage may be capable of movement in the x- and y-planes, or in the x-, y-, and z-planes. Sample platform 506 may be integrated with droplet generator 502 and/or optics system 508.

System 500 may include an electrical controller 515 to control the performance of the processes carried out within immunoassay dispensing instrument 501, immunoassay droplet generator 502, droplet ejector 503, sample platform 506 and/or optics system 508. For example, the controller may actuate droplet ejector 503 to control fluid flow, and eject a droplet 505 into the oil-filled chamber 504. In some examples, the electrical controller 515 may move the immunoassay droplet generator 502 after dispensing a droplet 505, and/or move the sample platform 506 to dispense fluid droplets in a preset pattern in oil 509 of chamber 504. In some examples, electrical controller 515 may communicate with circuitry 517. In some examples, the electrical controller 515 includes additional components which are not shown, such as controller circuitry, a processor, machine readable instructions, and other electronics for communicating with and controlling the components of system 500. The controller circuitry may receive data from a host system (not shown), such as a computer, and includes memory for temporarily storing data. The data may be sent to the system or a component thereof along an electronic, infrared, optical, or other information transfer path. A processor may be a central processing unit (CPU), a semiconductor-based microprocessor, a graphics processing unit (GPU), a microcontroller, special purpose logic hardware controlled by microcode or other hardware devices suitable for retrieval and/or execution of instructions stored in a memory, or combinations thereof. In addition to or alternatively to retrieving and executing instructions, the processor may include at least one integrated circuit (IC), other control logic, other electronic circuits, or combinations thereof that include a number of electronic components for performing the function. In some examples, the circuitry includes non-transitory computer-readable storage medium that is encoded with a series of executable instructions that may be executed by the processor. Non-transitory computer-readable storage medium may be an electronic, magnetic, optical, or other physical storage device that contains or stores executable instructions. Thus, non-transitory computer-readable storage medium may be, for example, Random Access Memory (RAM), an Electrically Erasable Programmable Read-Only Memory (EEPROM), a storage device, an optical disc, etc. In some examples, the computer-readable storage medium may be a non-transitory storage medium, where the term ‘non-transitory’ does not encompass transitory propagating signals.

FIGS. 5B, 5C and 5D show images of dispensed droplets in oil. FIGS. 5B, 5C and 5D show dispensed droplets, at 2×, 5×, and 10× magnification, respectively. Using an example immunoassay droplet generator consistent with the present disclosure, 9 pL droplets of an aqueous ink formulation were dispensed into an oil-filled chamber. The chamber included a volume of synthetic isoparaffinic hydrocarbon fluid sold under the trade name Isopar L. The droplets were dispensed into a planar array (300 dpi by 150 dpi), and did not entrap air in the oil although dispensed from a height of about 5 mm above the surface of the oil. The images show the droplets did not coalesce or spread after encapsulation in the oil.

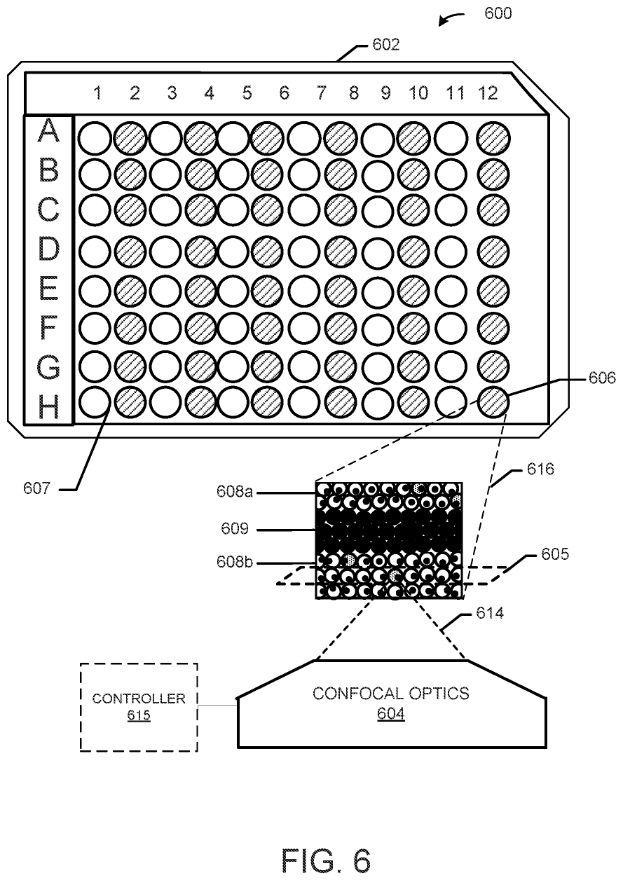

FIG. 6 illustrates another example implementation of a device for dispensing an immunoreactive magnetic particle and immunoassay reporter in a fluid droplet, consistent with the present disclosure. System 600 includes an immunoassay droplet generator (not shown), such as immunoassay droplet generator 502, or a device similar to the any of the devices described in FIGS. 1A-4. In the example of FIG. 6, system 600 includes a chamber 602 and confocal optics system 604.

Chamber 602 is shown as a 96-well microwell plate and includes selectively filled wells for compartmentalization of different populations of immunoassay droplets, with immunoassay-filled microwells 606 and empty microwells 607. A magnified horizontal section indicated by lines 616, show microwell 606 includes a high-density three dimensional array of dispensed droplets. A first row of immunoassay droplets 608a is separated from a second row of immunoassay droplets 608b by a row of separation fluid droplets 609. Shaded immunoassay droplets 608a and 608b include a generated signal.

Excitation light 614 may cause fluorescent signal molecules in an immunoassay droplet within optical section 605 to emit a detectable signal wavelength of light. Confocal optics system 604 may confine illumination and detection to a single, diffraction-limited, point within microwells of chamber 602. In some examples, the chamber may be scannable by confocal optics system 604. Points of light emitted from the scanned chamber may be detected by a PMT, for example, which is positioned behind a pinhole of confocal optics system 604. The output from the PMT may be built into an image. The excitation light 614 wavelength may vary based on the excitation spectrum of the generated signal. Light emitted by the generated signal is detected by a component of confocal optics system 604.

System 600 may include an electrical controller 615 for controlling the performance of the processes carried out within confocal optics system 604. For example, the controller may control image acquisition and/or analysis processes. The controller may include various unshown components such as circuitry, a processor, machine readable instructions, and other electronics for communicating with and controlling the components, as described above for system 500.

System 600 may have particular further utility for personal and point-of-care clinical diagnostics by increasing the number of droplets per unit volume, providing higher digitization and/or multiplexing capabilities, and high sensitivity using a relatively low cost consumable as an oil-filled chamber.

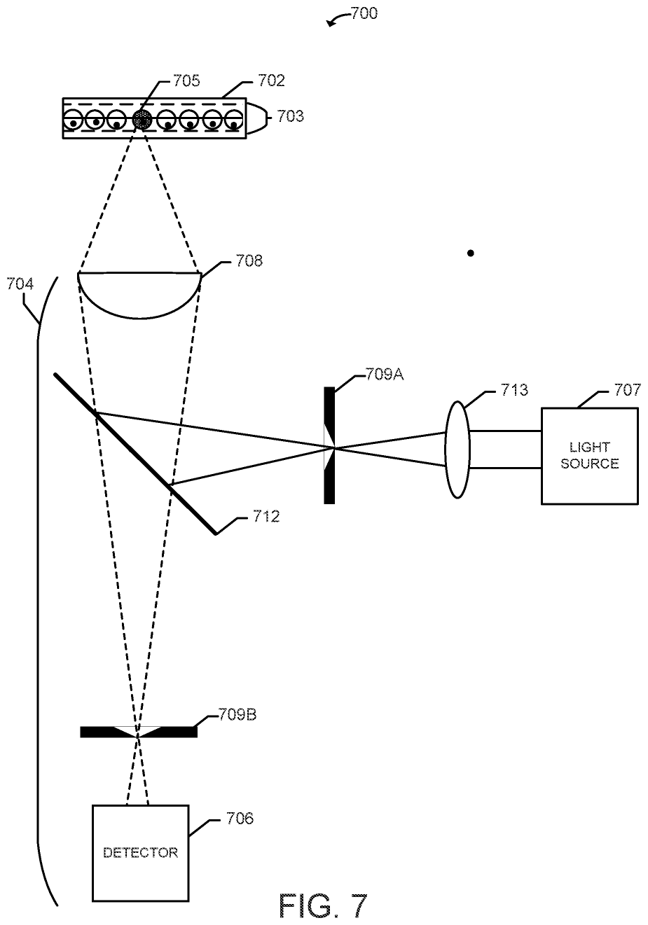

FIG. 7 illustrates an example confocal optics system 700 for incorporation into a system such as system 500 or 600, as described above. In the example of FIG. 7, section 702 shows a focal planes 703 including immunoassay droplets within optical section 605 as shown in FIG. 6. Immunoassay droplet 705 includes a generated signal. Confocal microscope configuration 704 may be used as a scanning confocal microscope. Configuration 704 includes light source 707, such as a laser excitation source, excitation filter 713, pinhole aperture 709A, mirror 712, objective 708, pinhole aperture 709B, and detector 706, such as a PMT detector. Light emitted by the source 707 passes through filter 713 and pinhole aperture 709A that is situated in a conjugate plane (confocal) with a scanning point on immunoassay droplet 705 and pinhole aperture 709B positioned in front of detector 706. As the light is reflected by mirror 712 and scanned across the specimen in the defined focal plane 703, secondary fluorescence emitted from points in the same focal plane pass back through mirror 712 and are focused as a confocal point at the detector pinhole aperture 709B. Refocusing objective 708 shifts the excitation and emission points to a new plane that becomes confocal with the pinhole apertures 709A and 709B of light source 707 and detector 706.

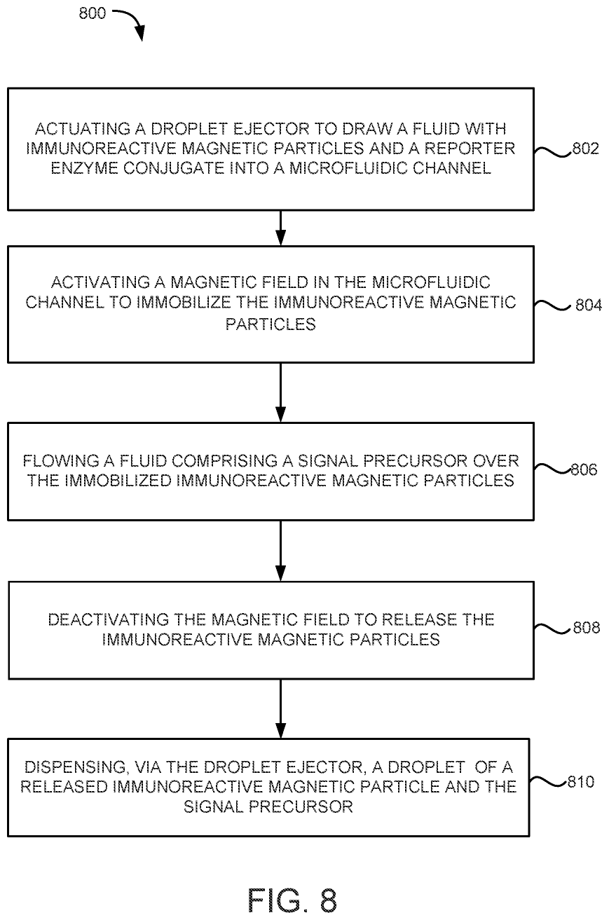

FIG. 8 is a flowchart of an example method 800 for dispensing an immunoreactive magnetic particle and immunoassay reporter in a fluid droplet, consistent with the present disclosure. In the example of FIG. 8, at 802, the method 800 includes actuating a droplet ejector coupled to a distal end of a microfluidic network to draw a fluid specimen comprising a suspension of immunoreactive magnetic particles and a reporter enzyme conjugate into a microfluidic channel. In some examples, actuating includes causing an operation of the droplet ejector. The droplet ejector may include a pump for drawing the fluid specimen into the microfluidic channel, such as an inertial pump. The droplet ejector may include a resistor. The resistor may be actuated by delivery of an electrical pulse, for example. The substrate may include the circuitry to operate or control operation of the droplet ejector.

A target antigen may be present in the fluid specimen, as discussed above. When the target antigen is present, the target antigen binds with the immunoreactive magnetic particles and the enzyme-conjugate to form an enzyme labeled immunocomplex in the fluid sample. As used herein, “reporter enzyme conjugate” refers to and includes an enzyme that is linked, joined, or connected with a target antigen binding moiety, which enzyme catalyzes a reaction that converts an immunoassay reporter to a detectable signal. The enzyme labeled immunocomplex may be an enzyme labeled immunocomplex as described for method 120.

In some examples, method 800 includes receiving, providing, or preparing the immunoreactive magnetic particles, the fluid specimen, and/or the reporter enzyme conjugate, as discussed above for method 120.

At 804, method 800 includes applying a magnetic field to a region of the microfluidic channel upstream of the droplet ejector to immobilize the immunoreactive magnetic particles on a surface of the microfluidic channel. A magnetic field may be applied from outside the microchannel by a magnet as described in method 120. The substrate may include circuitry to application of the magnetic field.

At 806, method 800 includes flowing a fluid comprising a signal precursor over the immobilized immunoreactive magnetic particles. The signal precursor may include an immunoassay reporter as described above. Non-immobilized components of the fluid specimen are removed from the region. The non-immobilized components may be ejected by the droplet ejector.

At 808, method 800 includes removing the magnetic field to release the immunoreactive magnetic particles. The magnetic field may be removed by moving an external magnet or by interrupting the electrical current. For example, removing the electric field may include switching an electromagnet off. In some examples, a resistor, micromixer, or micropump is actuated when the magnetic field is removed. The resistor, micromixer or micropump may be actuated to resuspend and/or agitate the released immunoreactive magnetic particles. Actuation of the resistor or micromixer may facilitate suspension of the immunoreactive magnetic particles in a volume of reporter fluid after deactivation of the magnetic field, and may assist dispersal of the immunoreactive magnetic particles within the microfluidic channel. For example, dispersal may separate the released immunoreactive magnetic particles into a discrete number of magnetic particles per droplet, improving assay sensitivity and precision of quantitative measurement.

At 810, method 800 includes dispensing a fluid droplet from the droplet ejector. The droplet ejector may include a piezoelectric device or a thermal resistor. The droplet ejector may include an open nozzle permitting a vapor bubble formed by the thermal resistor to eject a droplet of fluid into the ambient environment of the device. Use of a thermal resistor may achieve high speed generation of droplets. For example, a droplet generator including a thermal resistor may reduce the interval between droplets from about 30 minutes per droplet to about 3 minutes per droplet, as compared to a droplet generator operating at 1000 Hz. The fluid droplet comprises a portion of the released immunoreactive magnetic particles and the signal precursor. The droplet ejector may be capable of partitioning the released immunoreactive magnetic particles into a discrete number of immunoreactive magnetic particles, such as 0, 1, 2, 3, etc. immunoreactive magnetic particles per fluid droplet. The droplet ejector may be tuned to release a desired number of immunoreactive magnetic particles per droplet, such as for sensitive qualitative detection or precise quantitative measurement of a target antigen present in the sample fluid.

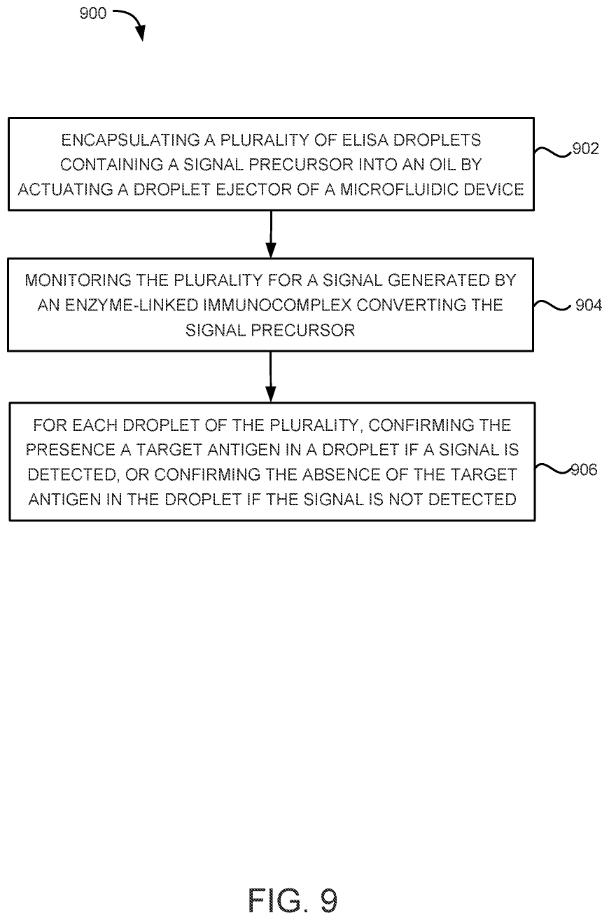

FIG. 9 is a flowchart of an example method 900 for dispensing a plurality of fluid droplets, consistent with the present disclosure. Droplet-based based methods may start with microliters of fluid and then dispense picoliters or nanoliters of fluid at specific locations. In the example of FIG. 9, at 902 method 900 includes encapsulating a plurality of ELISA droplets containing a signal precursor in oil by actuating a droplet ejector of a microfluidic device. The signal precursor may vary based on the reporter enzyme of the ELISA and/or the detector. In some cases, a suitable signal precursor may be any one of the immunoassay reporters described above, such as an immunoassay reporter as described with respect to method 120. The volume encapsulated may be picoliter scale, such as less than 10 picoliters, less than 20 picoliters, less than 30 picoliters, less than 40 picoliters, less than 50 picoliters.

In some examples, the oil may be contained within a chamber. Example chambers include, without limitation, 1-, 4-, and 8-well rectangular dishes, microwell plates, such as a 6-, 12-, 24-, 48-, 96-, 384-, or 1536-well plate, and a petri dish, such as a 50 mm, 100 mm, or a 300 mm petri dish. The chamber may be constructed from any material that is compatible with the oil and/or the method of reading the immunoassay results. In some examples, the chamber and/or oil may be selected from the oil filled chambers described with respect to FIG. 5A or 6, for example. In some examples, the oil may reduce the risk of droplet coalescence or collapse, with or without the use of surfactants. In some examples, the droplets may remain at the location into which they were dispensed for any duration such as an indefinite period of time, unless manipulated.

In some examples, the encapsulated ELISA droplets may be arranged in a multidimensional array, such as a planar (2D) or a three dimensional (3D) array. Compartmentalization may permit spatial organization of the dispensed droplets as in an array or a bulk emulsion. While bulk emulsions may be easily produced, multiplexed experiments involving droplets of different compositions, such as large-scale screening of biological samples, may involve indexing. In some examples, the array may be a high-density array such as an array of about 150 to about 1200 dots-per-inch (dpi), up to about 1×10⁶ dots/in², or up to about 1×10⁸ dots/in².

The droplet ejector may include a nozzle. In some examples, the nozzle may be positioned about 5 mm above the surface of the oil. The microfluidic device may include circuitry to control firing of the nozzle. For example, the circuitry may direct firing of the nozzle to eject each of the plurality of droplets at a target location. In some examples, positioning the nozzle may be achieved by XYZ-axis translation of the microfluidic device and/or the oil. In some examples, control of nozzle firing may enable array indexing and/or demultiplexing. Indexing permits droplets of interest to be identified and recovered based on location, for example. As used herein, “demultiplexing” refers to or includes assigning the location of an encapsulated ELISA droplet in the oil to its sample of origin based on nozzle firing data, such as the spatial data of nozzle and/or stage at the time of firing, or other variable.

The microfluidic device may be similar to a device as described above, such as device 100, 200, 300, or 400, for example, although the examples are not limited to these devices. Multiple microfluidic devices with a droplet generator may be used simultaneously. Parallel droplet encapsulation via actuation of a plurality of droplet generators may provide additional high-throughput processing and/or multiplexing capability.\

In some examples, method 900 includes partitioning the encapsulated droplets. Partitioning may include dividing the encapsulated ELISA droplets into discrete populations of ELISA droplets. Partitioning bulk samples into a large number of subsamples, allows detailed analysis of each subsample in an automated fashion for high-throughput analyses. A partitioned population may consist of droplets from a specific patient, or specific specimen type. Dividing may include encapsulating a droplet of a separator fluid after a predetermined number of ELISA droplets have been encapsulated. Partitioning may include encapsulating a separation fluid droplet in the oil adjacent to a droplet of the plurality. The separation fluid droplet may be dispensed by the microfluidic device. For example, after the last ELISA droplet of a first population has been dispensed, the droplet ejector may dispense a droplet of a separation fluid to delineate the terminus of the first population. The separator fluid may be a dye, ink, or any other fluid for marking a boundary of a population of ELISA droplets in the oil. In a multidimensional array, the oil may include a row and/or layer of encapsulated separator fluid droplets.

At 904, method 900 includes monitoring the plurality of ELISA droplets for a signal. The signal may be generated by conversion of the signal precursor when an enzyme-linked immunocomplex is dispensed in an ELISA droplet. In some examples, the enzyme-linked immunocomplex may include any one of the enzymes as described for method 120. Measuring a signal generated by a single enzyme molecule has utility in the early clinical diagnosis of various diseases, such as cancer, and to follow the efficacy of medical treatments, with potential for a point-of-care devices and personalized medicine.

Monitoring may include detecting a chromogenic, fluorescent or chemiluminescent signal produced by the enzyme-linked immunocomplex. Monitoring may be performed using a signal detector capable of detecting the generated signal. In some examples, the signal detector includes an optical sensor, contact image sensor (CIS), photodiode, LED, laser diode, photoresistor, photomultiplier tube (PMT), charge coupled device (CCD), complementary metal oxide semiconductor (CMOS), mirror or beamsplitter, lens, rod lens array, self-focusing lens array, optical filter, colorimeter, spectrophotometer, photodetector, fluorescence imager, plate reader, fluorescent microscope, confocal fluorimeter, camera, scanner, light or illumination source, such as an excitation source, laser, sample platform, movable stage, focus adjusting step motor, digital image processing system, or a combination thereof. Monitoring may include signal detection of single encapsulated ELISA droplet at a time, or wide-field analysis of a monolayer, for example.

At 906, method 900 includes confirming the presence or absence of the target antigen for each droplet. The presence of the target antigen is confirmed if the signal is detected. The absence of the target antigen may be confirmed if no signal is detected. Because mixing and mass transfer may be enhanced within a droplet, immunoassay results are available more rapidly than with prior ELISA methods. Droplets positive for the signal may be separated from negative droplets, or otherwise sorted, for further manipulation.

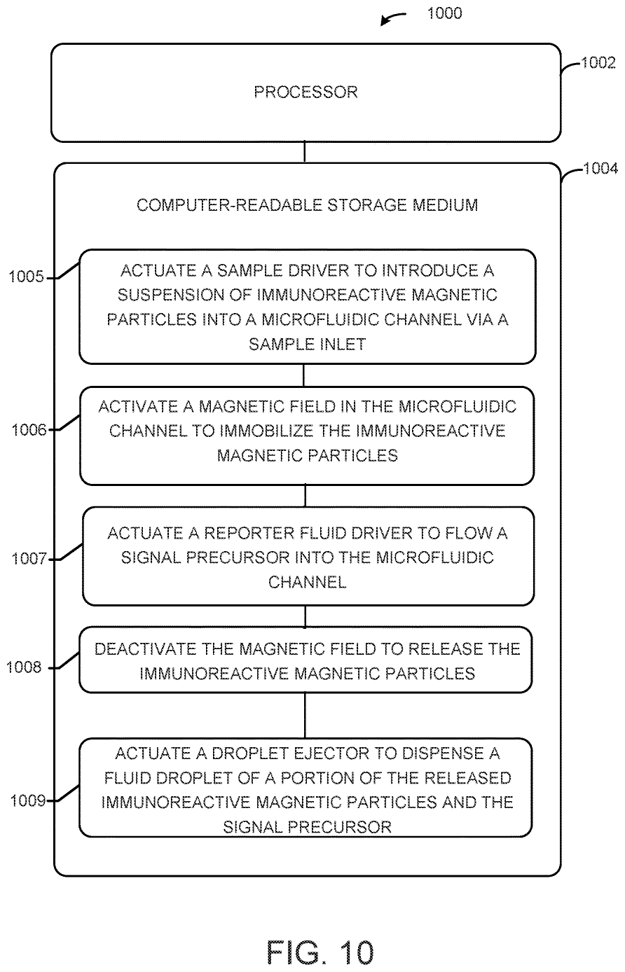

FIG. 10 is a block diagram of an example controller for dispensing an immunoreactive magnetic particle and immunoassay reporter in a fluid droplet including a processor and associated non-transient computer-readable medium containing instructions for the processor. In the example of FIG. 10, system 1000 includes a processor 1002, and a non-transitory computer-readable storage medium 1004. Non-transitory computer-readable storage medium 1004 includes instructions 1005, 1006, 1007, 1008, and 1009 for dispensing an immunoreactive magnetic particle and immunoassay reporter in a fluid droplet. System 1000 may be an immunoassay dispensing instrument an immunoassay dispensing instrument described above. For example, system 1000 may be immunoassay dispensing instrument 501 described in FIG. 5A. In some examples, system 1000 may be a printer, a mobile device, multimedia device, a secure microprocessor, a notebook computer, a desktop computer, an all-in-one system, a server, a network device, a controller, a wireless device, or any other type of device capable of executing the instructions 1005, 1006, 1007, 1008, and 1009, and in which, the previously described devices 100, 200, 300, or 400 for dispensing an immunoreactive magnetic particle and immunoassay reporter in a fluid droplet, may be inserted. In certain examples, system 1000 may include or be connected to additional components such as memory, controllers, etc. In some examples, the system 1000 may form part of the systems 500 and 600 of FIGS. 5A and 6.

Processor 1002 may be a central processing unit (CPU), a semiconductor-based microprocessor, a graphics processing unit (GPU), a microcontroller, special purpose logic hardware controlled by microcode or other hardware devices suitable for retrieval and execution of instructions stored in the non-transitory computer-readable storage medium 1004, or combinations thereof. The processor 1002 may fetch, decode, and execute instructions for dispensing an immunoreactive magnetic particle and immunoassay reporter in a fluid droplet, as previously discussed. As an alternative or in addition to retrieving and executing instructions, the processor 1002 may include at least one integrated circuit (IC), other control logic, other electronic circuits, or combinations thereof that include a number of electronic components for performing the functionality of instructions 1005, 1006, 1007, 1008, and 1009.

Non-transitory computer-readable storage medium 1004 may be an electronic, magnetic, optical, or other physical storage device that contains or stores executable instructions. Non-limiting examples of a non-transitory computer-readable storage medium 1004 include, for example, Random Access Memory (RAM), an Electrically Erasable Programmable Read-Only Memory (EEPROM), a storage device, an optical disc, etc. In some examples, the computer-readable storage medium 1004 may be a non-transitory storage medium, where the term ‘non-transitory’ does not encompass transitory propagating signals. As described in detail below, the non-transitory computer-readable storage medium 1004 may be encoded with a series of executable instructions. In some examples, non-transitory computer-readable storage medium 1004 may implement store and/or execute instructions 1005, 1006, 1007, 1008, and 1009.

Non-transitory computer-readable storage medium 1004 stores instruction 1005, which, when executed by processor 1002, may cause processor 1002 to control the actuation of a sample driver for introducing a suspension of enzyme-linked immunocomplexes into a microfluidic channel via a sample inlet. The actuation may be triggered by introduction of a sample fluid to a sample fluid feed slot, for example. Each enzyme-linked immunocomplex includes an immunoreactive magnetic particle, as described above. The suspension may include a composition such as a fluid sample, as described above. The sample driver may include an inertial pump. The sample driver may be positioned fluidically downstream from the sample inlet and or may be positioned within a wall of, or adjacent to, the sample inlet. The microfluidic channel may be supported by a substrate. The sample fluid inlet may be located in a sample fluid region.

Non-transitory computer-readable storage medium 1004 stores instruction 1006, which, when executed by processor 1002, may cause processor 1002 to activate a magnetic field for immobilizing the immunoreactive magnetic particles in the microfluidic channel. The magnetic field may be generated by a magnet. For example, processor 1002 may control a power supply connected to an electromagnet and/or control the position of a permanent magnet, relative to the microfluidic channel. The electromagnet and/or magnet may include any of the materials described above.

Non-transitory computer-readable storage medium 1004 stores instruction 1007, which, when executed by processor 1002, may cause processor 1002 to control the actuation of a reporter fluid driver for flowing a reporter fluid into the microfluidic channel. The reporter fluid includes a signal precursor, such as a signal precursor as described above. The reporter fluid driver may be a resistor. The resistor may be positioned within a wall of, or wall adjacent to, a reporter fluid inlet in fluidic communication with the microfluidic channel. Control may include actuating the reporter fluid driver in response to a change in the magnetic field. Control may include flowing a predetermined volume of reporter fluid into the microfluidic channel.

Non-transitory computer-readable storage medium 1004 stores instruction 1008, which, when executed by processor 1002, may cause processor 1002 to deactivate the magnetic field to release the immunoreactive magnetic particles in the microfluidic channel. Processor 1002 may control the power supply connected to the electromagnet and/or control the position of the permanent magnet, relative to the microfluidic channel, as described above. Processor 1002 may deactivate the magnetic field and control the reporter fluid driver to flow the reporter fluid to dilute the released immunoreactive magnetic particles.

Non-transitory computer-readable storage medium 1004 stores instruction 1009, which, when executed by processor 1002, may cause processor 1002 to control the actuation of a droplet ejector for dispensing a fluid droplet. The fluid droplet includes a portion of the released immunoreactive magnetic particles and they signal precursor. The droplet ejector may be positioned at a distal end of the microfluidic channel. The droplet ejector may include a resistor, such as a thermal resistor. The droplet ejector may include a nozzle. In some examples, control may include actuating the droplet ejector for dispensing a fluid droplet at a target location, for dispensing a predetermined volume of fluid, and/or when a discrete number of suspended immunoreactive magnetic particles flow proximate the fluid ejector, such as when one immunoreactive magnetic particle is proximate the fluid ejector.

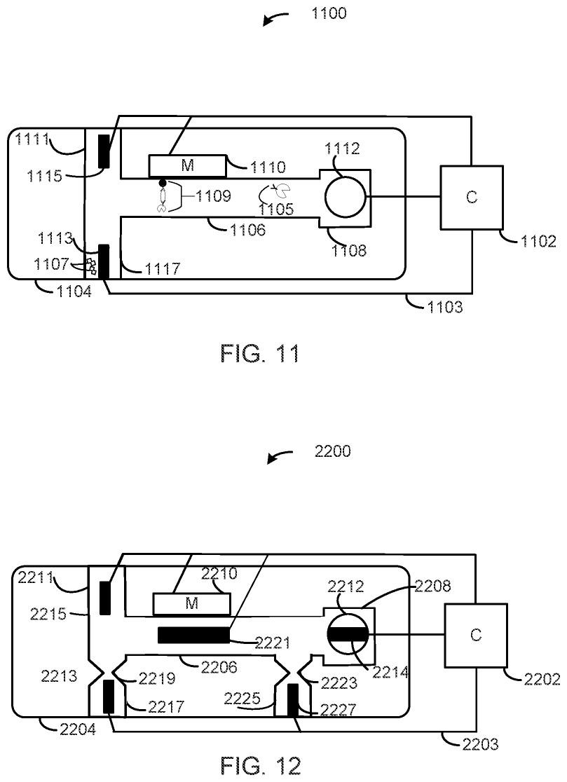

FIG. 11 describes an example system including a controller and circuitry for preparing and dispensing a fluid droplet of an enzyme-linked immunoassay containing an immunoreactive magnetic particle and the signal precursor, consistent with the present disclosure. Example system 1100, which is not drawn to scale, may be used to receive and flow a sample fluid containing an immunoreactive magnetic particle therethrough, immobilize the immunoreactive magnetic particle, and introduce a reporter fluid to the immobilized immunoreactive magnetic particle.

In the example of FIG. 11, system 1100 includes a microfluidic network 1106 supported by a substrate 1104, and a controller 1102. Substrate 1104 may be a substrate similar to a substrate as described above for device 100.

Controller 1102 includes circuitry 1103 to communicate with and control components of the microfluidic network. Microfluidic network 1106 includes a microfluidic channel 1108, a sample fluid inlet 1111 and a reporter fluid inlet 1117. Sample fluid inlet 1111 is to introduce a sample fluid to microfluidic channel 1108 of microfluidic network 1106, the sample fluid including enzyme-linked immunocomplex 1109 and an unbound reporter enzyme conjugate 1105. Reporter fluid inlet 1117 is to introduce a fluid comprising an immunoassay reporter 1107 to the microfluidic channel. Although a specific arrangement of microfluidic network 1106 is shown in FIG. 11, a system of the present disclosure may have other arrangements in other examples.

A magnet 1110 may be integrated with substrate 1104 within a region of the microfluidic channel downstream from sample fluid inlet 1111 and reporter fluid inlet 1117. In some examples, magnet 1110 is positioned adjacent to a wall of microfluidic channel 1108 to releasably immobilize the immunoreactive magnetic particle of in enzyme-linked immunocomplex 1109. As illustrated, magnet 1110 may be positioned adjacent to a side wall, yet other arrangements are within the scope of examples of the present disclosure. Magnet 1110 may include a magnet as described for device 100.

A sample fluid driver 1115 is positioned in sample fluid inlet 1111 and a reporter fluid driver 1113 is positioned in reporter fluid inlet 1117. Fluid driver 1113 and/or 1115 may be a micropump, such as a thermal micropump or a piezo-electric device. A thermal micropump may include a thin film resistor integrated with substrate 1104.

In some examples, a droplet ejector 1112 is coupled to a distal end of microfluidic channel 1108 to dispense a fluid droplet from the device. Droplet ejector 1112 may include a piezoelectric device or a thermal resistor to eject a droplet of fluid.

Circuitry 1103 operatively connects controller 1102 to reporter fluid driver 1113, sample fluid driver 1115, magnet 1110, and droplet ejector 1112. Circuitry 1103 may receive data from a host system, such as a computer operatively connected to controller 1102. Circuitry 1103 may be integrated with substrate 1104 or attached thereto.

Controller 1102 may selectively output control signals to selectively and independently actuate each fluid driver to cause fluid to flow into and through microfluidic channel 1108. Controller 1102 may selectively output control signals to selectively control magnet 1110 to generate or deactivate a magnetic field to draw, capture or release immunoreactive magnetic particles in the microfluidic channel, such as the immunoreactive magnetic particle of enzyme-linked immunocomplex 1109. Controller 1102 may selectively output control signals to selectively actuate droplet ejector 1112 to dispense a fluid droplet. For example, controller 1102 may selectively output control signals to independently actuate sample fluid driver 1115 to flow a sample fluid into microfluidic channel 1108, independently activate magnet 1110 to draw and capture immunoreactive magnetic particles present in the sample fluid, and independently actuate reporter fluid driver 1113 to flow a reporter fluid into the microfluidic channel, whereby non-immobilized components of the sample fluid, such as reporter enzyme conjugate 1105 are separated from the immobilized immunoreactive magnetic particles and immunoassay reporter 1107 may be brought into contact with immobilized enzyme-linked immunocomplex 1109. Thus, when controller 1102 selectively outputs control signals to magnet 1110 to release immunoreactive magnetic particles in the microfluidic channel, selective actuation of droplet ejector 1112 dispenses a fluid droplet containing an enzyme-linked immunocomplex 1109 and immunoassay reporter 1107.

FIG. 12 describes another example system including a controller and circuitry for dispensing a fluid droplet of an enzyme-linked immunoassay containing an immunoreactive magnetic particle and a signal precursor, consistent with the present disclosure. Example system 2200, which is not drawn to scale, may be used to receive and flow a sample fluid containing an immunoreactive magnetic particle therethrough, immobilize the immunoreactive magnetic particle, introduce a reporter fluid to the immobilized immunoreactive magnetic particle, and dispense a fluid droplet comprising the immobilized immunoreactive magnetic particle and the reporter fluid.

In the example of FIG. 12, system 2200 includes a microfluidic network 2206 supported by a substrate 2204, and a controller 2202. Substrate 2204 may be a substrate similar to a substrate as described above for system 1100. Controller 2202 includes circuitry 2203 to communicate with and control components of the microfluidic network. Microfluidic network 2206 includes a microfluidic channel 2208, a sample fluid inlet 2211, a reporter fluid inlet 2217, and a dilution fluid inlet 2225. Sample fluid inlet 2211 is to deliver a sample fluid to the microfluidic channel 2208. Although not illustrated in FIG. 12, the sample fluid includes an immunoreactive magnetic particle and optionally, an enzyme-linked immunocomplex and unbound reporter enzyme conjugates. Reporter fluid inlet 2217 is to deliver a fluid comprising an immunoassay reporter to the microfluidic channel 2208, and includes constriction 2219 to control fluid egress from microfluidic channel 2208 via reporter fluid inlet 2217. Dilution fluid inlet 2225 is to deliver a dilution fluid to microfluidic channel 2208, and includes constriction 2223 to control fluid egress from microfluidic channel 2208 via dilution fluid inlet 2225. Although a specific arrangement of microfluidic network 2206 is shown in FIG. 12, a system of the present disclosure may have other arrangements of inlets in other examples.

In some examples, a magnet 2210 is integrated with substrate 2204 within a region of the microfluidic channel downstream from sample fluid inlet 2211 and reporter fluid inlet 2217 and fluidically upstream from dilution fluid inlet 2225. Magnet 2210 may be positioned adjacent to a wall of microfluidic channel 2208 to releasably immobilize an immunoreactive magnetic particle of the sample fluid. Magnet 2210 may include a magnet as described for device 100.

In some examples, a sample fluid driver 2215 is positioned in sample fluid inlet 2211, a reporter fluid driver 2213 is positioned in reporter fluid inlet 2217, and a microchannel fluid driver 2221 is positioned in the region of microfluidic channel 2208 adjacent to magnet 2210. The fluid drivers 2213, 2215, and/or 2221 may include resistors, such as those previously described in connection with FIG. 1A. Fluid drivers 2213, 2215, and/or 2221 may be a micropump, such as a thermal micropump or a piezo-electric device. A thermal micropump may include a thin film resistor integrated with substrate 2204.

In some examples, a droplet ejector 2212 including resistor 2214 is coupled to a distal end of microfluidic channel 2208 to dispense a fluid droplet from microfluidic network 2206. Resistor 2214 may include thermal resistor to eject a droplet of fluid.

Circuitry 2203 operatively connects controller 2202 to magnet 2210, reporter fluid driver 2213, resistor 2214, sample fluid driver 2215, and microchannel fluid driver 2221. In some examples, circuitry 2203 may receive data from a host system, such as a computer operatively connected to controller 2202. In some examples, circuitry 2203 may be integrated with substrate 2204 or attached thereto.

In some examples, controller 2202 may selectively output control signals to selectively and independently actuate each fluid driver to cause fluid to flow into and through microfluidic channel 2208, and/or to facilitate resuspension of immunoreactive magnetic particles and/or to dilute resuspended immunoreactive magnetic particles. In some examples, controller 2202 may selectively output control signals to selectively activate magnet 2210 to generate or deactivate a magnetic field to draw, capture or release immunoreactive magnetic particles in the microfluidic channel. In some examples, controller 2202 may selectively output control signals to selectively actuate resistor 2214 to dispense a fluid droplet therefrom. For example, controller 2202 may selectively output control signals to independently actuate sample fluid driver 2215 to flow a sample fluid into microfluidic channel 2208, independently activate magnet 2210 to draw and capture immunoreactive magnetic particles present in the sample fluid, independently actuate reporter fluid driver 2213 to flow a reporter fluid through constriction 2219 into microfluidic channel 2208, whereby non-immobilized components of the sample fluid are separated from the immobilized immunoreactive magnetic particles and signal precursors may be brought into contact with any immobilized enzyme-linked immunocomplexes.

When controller 2202 selectively outputs control signals to magnet 2210 to release immunoreactive magnetic particles, controller 2202 may selectively output control signals to selectively actuate microchannel fluid driver 2221 to agitate the resuspended immunoreactive magnetic particles and flow the reporter fluid downstream to dilution fluid inlet 2225.

In some examples, controller 2202 may independently actuate dilution fluid driver 2227 to flow a dilution fluid through constriction 2223 into microfluidic channel 2208, whereby resuspended immunoreactive magnetic particles may be diluted and flowed downstream towards droplet ejector 2212. Controller 2202 may selectively output control signals to microchannel driver 2221 to dispense a fluid droplet of reporter fluid containing an immunoreactive magnetic particle. Controller 2202 may output control signals to selectively control the volume dispensed from droplet ejector 2212. In some examples, controller 2202 may selectively output control signals to microchannel driver 2221 to dispense a fluid droplet of reporter fluid containing a single immunoreactive magnetic particle. Single particle immunoassays may provide improved limits of detection of analytes present in low or very low concentrations in a sample fluid. Immunoassays capable of single particle analysis may facilitate earlier diagnosis of a medical condition, than prior ELISA platforms, for example.

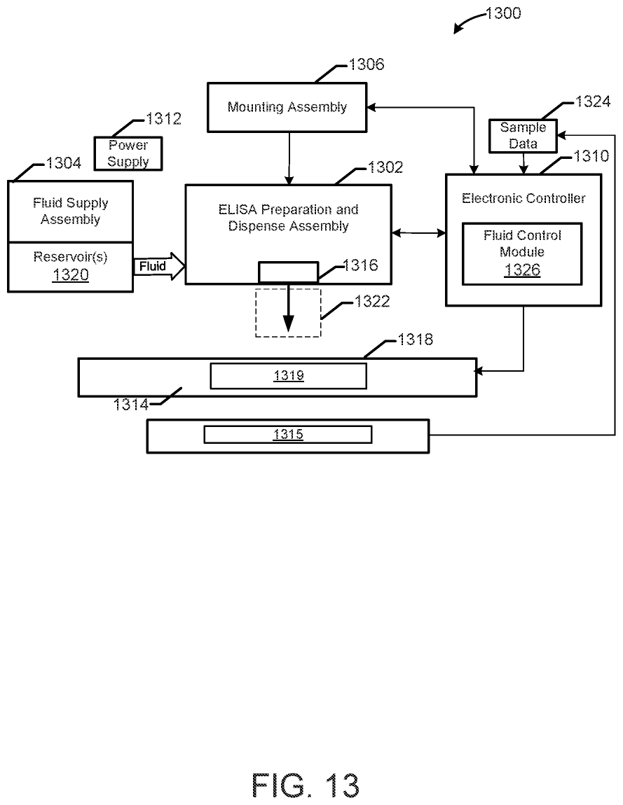

FIG. 13 is a block diagram illustrating an example system for dispensing an immunoreactive magnetic particle and immunoassay reporter in a fluid droplet. In the example of FIG. 13, system 1300 includes ELISA preparation and dispense assembly 1302, a fluid supply assembly 1304, a mounting assembly 1306, a droplet collection assembly 1318, supporting oil-filed chamber 1319, an electronic controller 1310, and a power supply 1312 that provides power to the various electrical components of system 1300. In some examples, ELISA preparation and dispense assembly 1302 having droplet ejector 1316 is a device for dispensing an immunoreactive magnetic particle and immunoassay reporter in a fluid droplet as described above. In some examples, ELISA preparation and dispense assembly 1302 may include components of, or be similar to device 100, 200, 300, or 400 or any one of systems 1000, 1100 and 2200. In some examples, ELISA preparation and dispense assembly 1306 may be an immunoassay dispensing instrument as described above, such as immunoassay dispensing instrument 501. ELISA preparation and dispense assembly 1306, a fluid supply assembly 1304, a mounting assembly 1306, electronic controller 1310, and one power supply 1312 may be integrated into an immunoassay dispensing instrument into which a substrate comprising a microfluidic network and a droplet ejector may be inserted.

ELISA preparation and dispense assembly 1302 includes at least one droplet ejector 1316 that ejects drops of ELISA toward the oil-filed chamber 1319 of droplet collection assembly 1318. The oil-filed chamber may be similar to the oil-filled chambers, described above. In some examples, droplet collection assembly 1318 and ELISA preparation and dispense assembly 1302 are movable relative to each other.

In some examples, fluid supply assembly 1304 may supply fluid to ELISA preparation and dispense assembly 1302 and may include a reservoir 1320 to store fluid. The fluid may be a sample fluid, a reporter fluid, a dilution fluid, and/or a reporter fluid, and reservoir 1320 may include respective reservoirs for each fluid.

In some examples, fluid supply assembly 1304 and ELISA preparation and dispense assembly 1302 are housed together in dispense cartridge, such as an inkjet cartridge. Fluid supply assembly 1304 may be housed separately from ELISA preparation and dispense assembly 1302 and supplies fluid via an interface connection, such as supply tubing. Reservoir 1320 may be removable, replaceable, and/or refillable.

In some examples, mounting assembly 1306 and droplet collection assembly 1318 may be integrated or physically separate components. Mounting assembly 1306 may position ELISA preparation and dispense assembly 1302 relative to droplet collection assembly 1318, and droplet collection assembly 1318 may position an oil-filed chamber thereon relative to ELISA preparation and dispense assembly 1302. Thus, a droplet ejection zone 1322 is defined adjacent to droplet ejector 1316 in an area between the ELISA preparation and dispense assembly 1302 and the oil-filled chamber. In some examples, mounting assembly 1306 includes a carriage to move ELISA preparation and dispense assembly 1302 relative to droplet collection assembly 1318. In other examples, ELISA preparation and dispense assembly 1302 is fixed at a prescribed position relative to droplet collection assembly 1318. Thus, droplet collection assembly 1318 positions the oil-filled chamber relative to ELISA preparation and dispense assembly 1302. In some examples, droplet collection assembly 1318 includes a stage such as described for sample platform 506 of FIG. 5A.

Electronic controller 1310 may include a processor, firmware, software, memory component(s) including volatile and non-volatile memory components, and other electronics to communicate with and control positions ELISA preparation and dispense assembly 1302, mounting assembly 1306, and droplet collection assembly 1318. Electronic controller 1310 may receive data 1324 from a host system, such as a computer, and temporarily stores data 1324 in a memory. Data 1324 may be sent to system 1300 along an electronic, infrared, optical, or other information transfer path. Data 1324 represents, for example, a target locations to dispense the droplets to be ejected, and may include job commands and/or command parameters related to the ELISA samples to be analyzed. In some cases, electronic controller 1310 includes fluid control module 1326 stored in a memory of controller 1310. Fluid control module 1326 executes on electronic controller 1310, such as a processor thereof, to control the activation sequence of droplet ejection elements, magnetic field elements, and fluid driver elements within ELISA preparation and dispense assembly 1302, as well as the time interval between such activations. Thus, fluid control module 1326 includes instructions to be executed by electronic controller 1310.

In some examples, ELISA preparation and dispense assembly 1302 includes multiple devices having a microfluidic network and droplet ejector for dispensing an immunoreactive magnetic particle and immunoassay reporter in a fluid droplet to eject ELISA droplets prepared form various different samples simultaneously or concurrently at respective target locations. For example, two devices, each having a droplet ejector may dispense ELISA droplets in an oil-filled chamber, concurrently in parallel rows.

In some examples, system 1300 includes monitoring assembly 1314. Monitoring assembly 1314 may include optics system 1315. The optics system 1315 may include any component of optics system 508 of FIG. 5A, confocal optics system 604 of FIG. 6, or confocal microscope configuration 704 of FIG. 7, for example. Monitoring assembly 1314 and optics system 1315 may be integrated with each other or another component of system 1300. For example, monitoring assembly 1314 and droplet collection assembly 1318 may be integrated or may be physically separate components. Droplet collection assembly 1318 may position the oil-filled chamber 1319 relative to monitoring assembly 1314.

Electronic controller 1310 may include a processor, firmware, software, memory components including volatile and non-volatile memory components, and other electronics to communicate with and control position of the monitoring assembly 1314, or a component thereof, or to control the position of the droplet collection assembly 1318. Electronic controller 1310 may receive data 1324 from a component of the monitoring assembly 1314, such as a computer, and temporarily store data 1324 in a memory. Data 1324 may be transmitted from the monitoring assembly 1314 to electronic controller 1310 or a host computer, along an electronic, infrared, optical, or other information transfer path. Thus, data 1324 may include for example, a location of a droplet producing or emitting a detectable signal within the oil-filled chamber, or a wide-field image of a plane of the oil-filled chamber showing both droplets that are positive and droplets that are negative for a detectable signal. Monitoring assembly 1314 may include a signal detector having an optical sensor, contact image sensor (CIS), photodiode, LED, laser diode, photoresistor, photomultiplier tube (PMT), charge coupled device (CCD), complementary metal oxide semiconductor (CMOS), lens, mirror or beamsplitter, rod lens array, self-focusing lens array, optical filter, colorimeter, spectrophotometer, photodetector, fluorescence imager, plate reader, fluorescent microscope, confocal fluorimeter, camera, scanner, light or illumination source, such as an excitation source, laser, sample platform, movable stage, focus adjusting step motor, digital image processing system, or combination thereof.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A device comprising:
a substrate;
a microfluidic network supported by the substrate, the microfluidic network comprising:
a microfluidic channel,
a sample fluid channel to deliver a sample fluid comprising an immunoreactive magnetic particle to the microfluidic channel, the immunoreactive magnetic particle including a capture antibody to bind to a target antigen present in the sample fluid, and
a reporter fluid channel to deliver a fluid comprising an immunoassay reporter to the microfluidic channel;
a magnet integrated with the substrate within a region of the microfluidic channel downstream from the sample fluid channel and the reporter fluid channel, wherein the magnet is positioned adjacent to a wall of the microfluidic channel; and,
a droplet ejector coupled to a distal end of the microfluidic channel to dispense a fluid droplet comprising the immunoreactive magnetic particle and the immunoassay reporter.

2. The device of claim 1, wherein the droplet ejector comprises a thermal resistor.

3. The device of claim 1, the microfluidic network comprising a wash fluid channel in fluidic communication with the microfluidic channel upstream of the region, wherein the wash fluid channel comprises a constriction and a resistor to pump wash fluid through the constriction.

4. The device of claim 1, the microfluidic network comprising a dilution fluid channel in fluidic communication with the microfluidic channel at a location proximate the droplet ejector.

5. The device of claim 1, wherein the substrate comprises a resistor to agitate immunoreactive magnetic particle in the region of the microfluidic channel adjacent to the magnet.

6. A method comprising:
actuating a droplet ejector coupled to a distal end of a microfluidic network to draw a fluid specimen comprising a suspension of immunoreactive magnetic particles and a reporter enzyme conjugate into a microfluidic channel, wherein if a target antigen is present, the target antigen, an immunoreactive magnetic particle, and the reporter enzyme conjugate form an enzyme-linked immunocomplex in the fluid specimen;
applying a magnetic field in a region of the microfluidic channel upstream of the droplet ejector to immobilize the immunoreactive magnetic particles on a surface of the microfluidic channel;
flowing a fluid comprising a signal precursor over the immobilized immunoreactive magnetic particles, whereby non-immobilized components of the fluid specimen are removed from the region;
removing the magnetic field to release the immunoreactive magnetic particles; and
dispensing a fluid droplet from the droplet ejector, the fluid droplet comprising a portion of the released immunoreactive magnetic particles and the signal precursor.

7. The method of claim 6, comprising monitoring the dispensed fluid droplet for a signal catalyzed by the enzyme-linked immunocomplex, wherein detection of the signal indicates the target antigen is present in the fluid specimen.

8. The method of claim 7, wherein monitoring comprises detecting a chromogenic, fluorescent or chemiluminescent signal produced by the enzyme-linked immunocomplex.

9. The method of claim 6, wherein the fluid droplet is dispensed into a chamber filled with an oil immiscible with the fluid droplet.

10. The method of claim 9, wherein a plurality of droplets are dispensed in the oil in a planar array or a three dimensional array.

11. The method of claim 10, comprising partitioning the dispensed droplets.

12. The method of claim 11, wherein partitioning comprises dispensing a separation fluid droplet in the oil adjacent to a droplet of the plurality.

13. A system comprising:
an immunoassay droplet generator comprising:
a microfluidic network comprising:
a microfluidic channel to receive a sample fluid comprising a set of enzyme-linked immunocomplexes comprising an immunoreactive magnetic particle, the microfluidic channel comprising a region to release capture the immunoreactive magnetic particles in response to a magnetic field;
a signal precursor fluid channel in fluidic communication with the microfluidic channel at a position fluidically upstream of the region to releasably capture the immunoreactive magnetic particles and to introduce a signal precursor to the enzyme-linked immunocomplexes; and
an outlet coupled to a distal end of the microfluidic channel comprising a droplet ejector to dispense an immunoassay droplet; and,
an oil-filled chamber positioned to receive the dispensed immunoassay droplet, wherein the oil is immiscible with the immunoassay droplet.

14. The system of claim 13, comprising a signal detector.

15. The system of claim 14, wherein the signal detector comprises a confocal point fluorimeter.
